SEQUENCE LISTING (18 sequences)

SEQ ID NO: 1 - DNA, 838 bp, Homo sapiens
attttgcgtggctaaaaagcagagccatgccgctggggaagcagttgcgatttagccatggctgcagcttggaccgtggtgctggtgactttggtgctaggcttggccgtggcaggccctgtccccacttccaagcccaccacaactgggaagggctgccacattgacaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctgacaccccacaccttatttatgcgctgagccctactccttccttaatttatttcctctcaccctttatttatgaagctgcagccctgactgagacatagggctgagtttattgttttacttttatacattatgcgcaaataaacaacaaggaattggaaaaaaaaaaaaaaaaaaaaaaa

SEQ ID NO: 2 - protein, 200 aa, Homo sapiens
MAAAWTVVLVTLVLGLAVAGPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 3 - DNA, 1088 bp, Homo sapiens
cagaaagagtcaaagccaggacacagtctgagatccagaagaggggactgaaaagaacagagactccagacaagacccaaacagaccctgggtgacagcctcagagtgtttcttctgctgacaaagaccagagatcaggaatgaaactagacatgaccggggactgcatgccagtgctggtgctgatggccgcagtgctgaccgtgactggagcagttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctgacccttccgccagtcatgcaacctgagattttatttataaattagccacttggcttaatttattgtcacccagtcgctatttatgtatttgtgtatgtaaatccaactcacctccaggaaaatgtttatttttctactttttgaaatccttgttgaaataaacaatgaggaaaagacacccatgacgtgggactgtgtgtgcgttggtgtgtatttcctttgcattgctgccataacaaattaccctaaaagtagcatctagaacagcaggttcattgagtctgtgctgtccactggggtccccaggtcacatgtcactatcgagcacctggaatgtaggtggtgc

SEQ ID NO: 4 - protein, 200 aa, Homo sapiens
MKLDMTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 5 - DNA, 828 bp, Homo sapiens
cagaaagagtcaaagccaggacacagtctgagatccagaagaggggactgaaaagaacagagactccagacaagacccaaacagaccctgggtgacagcctcagagtgtttcttctgctgacaaagaccagagatcaggaatgaaactagacatgactggggactgcacgccagtgctggtgctgatggccgcagtgctgaccgtgactggagcagttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctgaccctcccaccagtcatgcaacctgagattttatttataaattagccacttgtcttaatttattgccacccagtcgctatttatgt

SEQ ID NO: 6 - protein, 200 aa, Homo sapiens
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 7 - DNA, 300 bp, Homo sapiens
  misc_feature (300)..(300): any nucleotide
gcccttgaagtgtcgcttctgctgaaggactgcaggtgccgctcccgcctcttccccaggacctgggacctgaggcagctgcaggtgagggagcgccctgtggctttggaggctgagctggccctgacactgaaggtcctggaggtcaccgctgatgctgatccggccctgggggatgtcctggaccagctccttcacaccctccacaacatcctctcccagcttggggccagtatccagcctcagcaaggcccaggccccggggccgcctccaccactggctgcaccagctcaagggcn

SEQ ID NO: 8 - protein, 100 aa, Homo sapiens
ALEVSLLLKDCRCRSRLFPRTWDLRQLQVRERPVALEAELALTLKVLEVTADADPALGDVLDQLLHTLHNILSQLGASIQPQQGPGPGAASTTGCTSSRA

SEQ ID NO: 9 - DNA, 129 bp, Mus musculus
atggctacagtgtgcctgctgggtctggtgaccatggtgctgggcttggccacagctggctctgtccctccttccaagcccaccccaaccggatcaggctgtcaccttggtagtttccagtctctgtca

SEQ ID NO: 10 - protein, 43 aa, Mus musculus
MATVCLLGLVTMVLGLATAGSVPPSKPTPTGSGCHLGSFQSLS

SEQ ID NO: 11 - DNA, 936 bp, Mus musculus
tctcgccacaccctgctctgggcttccccagcctgggctccctagtggcaggtatcaacctgctaccttattttcacttttcctacatcagctggggctgcccatcagaccaggttaaaagcatggagcacggatggcagtgcactccacagagctggaaactcagagcctcagtcctcacacagcaacaggccacaggggacgacccaggccagagacaccaagcaagaaccagagaaaacctcaagggagacctgagtccctatctcctcacagaccccggagagcaacatgaagccagaaacagctgggggccacatgctcctcctgctgttgcctctgctgctggccgcagtgctgacaagaacccaagctgaccctgtccccagggccaccaggctcccagtggaagcaaaggattgccacattgctcagttcaagtctctgtccccaaaagagctgcaggccttcaaaaaggccaaggatgccatcgagaagaggctgcttgagaaggacctgaggtgcagttcccacctcttccccagggcctgggacctgaagcagctgcaggtccaagagcgccccaaggccttgcaggctgaggtggccctgaccctgaaggtctgggagaacatgactgactcagccctggccaccatcctgggccagcctcttcatacactgagccacattcactcccagctgcagacctgtacacagcttcaggccacagcagagcccaggtccccgagccgccgcctctcccgctggctgcacaggctccaggaggcccagagcaaggagacccctggctgcctggaggcctctgtcacctccaacctgtttcgcctgctcacccgggacctcaagtgtgtggccaatggagaccagtgtgtctgacctggaaccctcctgccagcttcaggaattcttcac

SEQ ID NO: 12 - protein, 202 aa, Mus musculus
MKPETAGGHMLLLLLPLLLAAVLTRTQADPVPRATRLPVEAKDCHIAQFKSLSPKELQAFKKAKDAIEKRLLEKDLRCSSHLFPRAWDLKQLQVQERPKALQAEVALTLKVWENMTDSALATILGQPLHTLSHIHSQLQTCTQLQATAEPRSPSRRLSRWLHRLQEAQSKETPGCLEASVTSNLFRLLTRDLKCVANGDQCV

SEQ ID NO: 13 - DNA, 18 bp, Artificial Sequence
  oligonucleotide primer
aggccctgtccccacttc

SEQ ID NO: 14 - DNA, 22 bp, Artificial Sequence
  oligonucleotide primer
gagatttgaacctgccaatgtg

SEQ ID NO: 15 - DNA, 23 bp, Artificial Sequence
  oligonucleotide probe
cccaccacaactgggaagggctg

SEQ ID NO: 16 - DNA, 20 bp, Artificial Sequence
  oligonucleotide primer
ggagctgcaggcctttaaga

SEQ ID NO: 17 - DNA, 18 bp, Artificial Sequence
  oligonucleotide primer
gcggcacttgcagtcctt

SEQ ID NO: 18 - DNA, 31 bp, Artificial Sequence
  oligonucleotide probe
ccaaagatgccttagaagagtcgcttctgct

(19) United States
(12) Patent Application Publication
(10) Pub. No.: US 2007/0081973 A1
(43) Pub. Date: Apr. 12, 2007

(54) Cytokine polypeptides

(75) Inventors: Peter R. Baum, Seattle, WA (US); Bruce A. Mosley, Seattle, WA (US); Randal R. Ketchem, Snohomish, WA (US); Scott L. Taylor, Seattle, WA (US)

Correspondence Address: IMMUNEX CORPORATION;LAW DEPARTMENT, 1201 AMGEN COURT WEST, SEATTLE, WA, 98119, US

(73) Assignee: Immunex Corporation, Thousand Oaks, CA (US)

(21) Appl. No.: 11/602,056

(22) Filed: Nov. 20, 2006

Related U.S. Application Data
(63) Continuation of application No. 10/142,717, filed on May 8, 2002, now abandoned.
(60) Provisional application No. 60/290,239, filed on May 10, 2001.

(51) Int. Cl. A61K 38/19; C07H 21/04; C12P 21/02; A61K 39/395; C07K 14/52; C07K 16/24

(52) U.S. Cl. 424/85.1; 435/69.5; 435/320.1; 435/325; 530/351; 530/388.22; 424/145.1; 536/23.5

(57) ABSTRACT

This invention relates to IMX129840 cytokines, including new mammalian cytokine polypeptides; to methods of making such polypeptides; to methods of using them to treat conditions and diseases involving proliferation and/or differentiation of cells from pluripotent stem cell precursors; and to methods of identifying compounds that alter IMX129840 cytokine polypeptide activities.

This application is a continuation of U.S. patent application Ser. No. 10/142,717, filed May 8, 2002, which claims the benefit under 35 U.S.C. 119(e) of U.S. provisional application Ser. No. 60/290,239, filed 10 May 2001, which is incorporated in its entirety by reference herein.

### FIELD OF THE INVENTION

This invention relates to IMX129840 cytokine polypeptides, IMX129840-1, -2, -3 and -4, including new members of the cytokine polypeptide family, and to methods of making and using IMX129840 cytokine polypeptides.

### BACKGROUND OF THE INVENTION

The cytokine polypeptides are a related group of secreted polypeptides, having a three-dimensional structure characterized by a ‘bundled’ arrangement of four alpha helices. Members of this family of “four-alpha-helical-bundle” (4AHB) polypeptides also include hematopoietic growth factors, interferons, and hormones. The 4AHB cytokine polypeptides are all involved in regulating either the proliferation or the development of cells such as hematopoietic cells or immune cells from pluripotent stem cell precursors, with different combinations of cytokines affecting the formation of different cell types such as T cells, B cells, erythrocytes, megakaryocytes, mast cells, eosinophils, neutrophils, monocytes, macrophages, dendritic cells, and osteoclasts. However, some subgroups of these cytokines also affect biological activities of cells outside the hematopoietic or immune cell system, with their receptors widely expressed in different tissues (Nicola and Hilton, 1999, Advances in Protein Chemistry 52: 1-65).

Structural features of the cytokine family of polypeptides that are commonly, but not universally, present include signal sequences directing movement of the cytokine precursor polypeptide through the cell membrane to produce a secreted cytokine, or to a topologically exterior surface of the cell membrane to produce a membrane-bound form of the cytokine that is then proteolytically cleaved and released from the cell. While most members of the 4AHB cytokine family are active as monomeric molecules, some form functional homodimers, or interact with soluble forms of cytokine receptors to form a heterodimeric molecule (Nicola and Hilton, 1999, Advances in Protein Chemistry 52: 1-65). Cysteine residues in 4AHB cytokines have been found to be involved in intramolecular disulfide bonds that stabilize cytokine structure, for example in the case of leptin/OB (Rock et al., 1996, Horm Metab Res 28: 649-652), or in intermolecular disulfide bonds related to multimer formation, such as in M-CSF (Pandit et al., 1992, Science 258: 1358-1362). The four alpha helices of the 4AHB cytokines, helices A through D, are arranged in an “up up down down” configuration (Kallen et al., 1999, J Biol Chem 274: 11859-11867). The A and D helices of the interleukin-6 (IL-6) cytokine have been found to include hydrophobic residues important in forming hydrophobic binding interactions with the IL-6 receptor alpha chain, interspersed with charged residues that are believed to form salt-bridge clusters with charged residues on the receptor chain, shielding the nearby hydrophobic residues from water molecules and stabilizing the cytokine-receptor interactions (Grötzinger et al., 1997, PROTEINS: Structure, Function, and Genetics 27: 96-109). The results of mutational studies identifying functional residues in the A and D helices of thrombopoietin (TPO), a hematopoietic cell growth factor of the 4AHB cytokine family (Jagerschmidt et al., 1998, Biochem J 333: 729-734), are consistent with this model of cytokine-receptor interaction.

Structurally, the 4AHB cytokine family can be divided into two groups: “short-chain” cytokines with shorter core alpha helices and two-strand beta-sheet structures in the inter-helical loops, and “long-chain” cytokines with longer core alpha helices and in many cases shorter alpha helices in the loop regions. The 4AHB cytokine family can also be subdivided on the basis of the type(s) of receptor complex(es) they interact with. For example, 4AHB cytokines may bind to a Type I or a Type II cytokine receptor which propagate regulatory signals through various members of the JAK and STAT families of intracellular signaling molecules, or they may bind to receptors with intrinsic tyrosine kinase activities (Nicola and Hilton, 1999, Advances in Protein Chemistry 52: 1-65); further, a variety of functional conformations are observed among the receptors for 4AHB cytokines, such as single-chain receptors, homodimers, heterodimers of an alpha ‘cytokine-binding’ chain and a beta ‘signaling’ chain that may also be present (‘shared’) in receptor complexes for other cytokines, and receptor complexes with three or more receptor chains (Cosman, 1993, Cytokine 5: 95-106).

Because of their roles in differentiation of hematopoietic and immune cells, 4AHB cytokine polypeptides are involved in a wide range of biological processes and associated disease states and conditions. For example, interaction of the 4AHB cytokine erythropoietin (EPO) with its receptor (a homodimer with an intracellular signaling domain that activates a pathway including JAK2 and STAT5) stimulates the proliferation and differentiation of erythrocyte precursor cells in adults, making EPO useful for treating anemia. The 4AHB cytokines thrombopoietin (TPO) and Granulocyte Colony-Stimulating Factor (G-CSF) also have hematopoiesis-stimulating activity. Other biological effects of 4AHB cytokines include regulation of neural cell and keratinocyte development, regulation of whole-body metabolism (an effect demonstrated by growth hormone (GH), prolactin (PRL), and leptin/OB, for example); stimulation of a proinflammatory response to infection or injury and of innate immunity (Granulocyte-Macrophage Colony-Stimulating Factor (GM-CSF), IL-3, IL-5, IL-6, oncostatin M (OSM), and leukemia inhibitory factor (LIF), for example); anti-viral activity (interferons such as interferon alpha, beta, and gamma); and stimulation of acquired immunity and driving the differentiation of helper T cells toward Th1 cell fates (IL-12) or Th2 cell fates (IL-2, IL-4, and IL-15, for example) (Nicola and Hilton, 1999, Advances in Protein Chemistry 52: 1-65).

In order to develop more effective treatments for conditions and diseases involving the proliferation or the development of cells from pluripotent stem cell precursors, information is needed about previously unidentified members of the 4AHB cytokine polypeptide family.

### SUMMARY OF THE INVENTION

The present invention is based upon the discovery of new human cytokine family members, IMX129840-1, IMX129840-2, IMX129840-3, and IMX129840-4.

The invention provides an isolated polypeptide consisting of, consisting essentially of, or more preferably, comprising an amino acid sequence selected from the group consisting of:

(a) the amino acid sequence of SEQ ID NO: 2;

(b) the amino acid sequence of SEQ ID NO: 4 or of SEQ ID NO: 6;

(c) an amino acid sequence that begins between amino acid A through B and ends between amino acid Y through Z, wherein sets of values for A, B, Y, and Z are selected from the group consisting of: A=44, B=47, Y=59, and Z=61 of SEQ ID NO:2; A=51, B=54, Y=66, and Z=68 of SEQ ID NO:4 or of SEQ ID NO:6; A=89, B=91, Y=102, and Z=112 of SEQ ID NO:2; A=96, B=98, Y=109, and Z=121 of SEQ ID NO:4 or of SEQ ID NO:6; A=111, B=116, Y=131, and Z=135 of SEQ ID NO:2; A 120, B=125, Y=140, and Z=144 of SEQ ID NO:4 or of SEQ ID NO:6; A=143, B=146, Y=157, and Z=159 of SEQ ID NO:2; and A=152, B=155, Y=166, and Z=168 of SEQ ID NO:4 or of SEQ ID NO:6;

(d) a fragment of an amino acid sequence of any of (a)-(c) comprising at least 20 contiguous amino acids;

(e) a fragment of an amino acid sequence of any of (a)-(c) comprising at least 30 contiguous amino acids;

(f) a fragment of an amino acid sequence of any of (a)-(c) having IMX129840 cytokine polypeptide activity;

(g) a fragment of an amino acid sequence of any of (a)-(c) comprising Helix A and/or Helix D amino acid sequences;

(h) amino acid sequences comprising at least 20 amino acids and sharing amino acid identity with the amino acid sequences of any of (a)-(g), wherein the percent amino acid identity is selected from the group consisting of: at least 70%, at least 75%, at least 80%, at least 85%, at least 90%, at least 95%, at least 97.5%, at least 99%, and at least 99.5%;

(i) an amino acid sequence of (h), wherein a polypeptide comprising said amino acid sequence of (h) binds to an antibody that also binds to a polypeptide comprising an amino acid sequence of any of (a)-(g); and

(j) an amino acid sequence of (h) or (i) having IMX129840 cytokine polypeptide activity.

Other aspects of the invention are isolated nucleic acids encoding polypeptides of the invention, with a preferred embodiment being an isolated nucleic acid consisting of, or more preferably, comprising a nucleotide sequence selected from the group consisting of:

(a) nucleotides 58 through 657 SEQ ID NO:1;

(b) nucleotides 141 through 740 of SEQ ID NO:3;

(c) nucleotides 141 through 740 of SEQ ID NO:5; and

(d) variants of (a)-(c).

An additional preferred embodiment of the invention is an isolated nucleic acid consisting of, or more preferably, comprising a nucleotide sequence selected from the group consisting of SEQ ID NO:1, SEQ ID NO:3, and SEQ ID NO:5.

The invention also provides an isolated genomic nucleic acid corresponding to the nucleic acids of the invention.

Other aspects of the invention are isolated nucleic acids encoding polypeptides of the invention, and isolated nucleic acids, preferably having a length of at least 15 nucleotides, that hybridize under conditions of moderate stringency to the nucleic acids encoding polypeptides of the invention. In preferred embodiments of the invention, such nucleic acids encode a polypeptide having IMX129840 cytokine polypeptide activity, or comprise a nucleotide sequence that shares nucleotide sequence identity with the nucleotide sequences of the nucleic acids of the invention, wherein the percent nucleotide sequence identity is selected from the group consisting of: at least 70%, at least 75%, at least 80%, at least 85%, at least 90%, at least 95%, at least 97.5%, at least 99%, and at least 99.5%.

Further provided by the invention are expression vectors and recombinant host cells comprising at least one nucleic acid of the invention, and preferred recombinant host cells wherein said nucleic acid is integrated into the host cell genome.

Also provided is a process for producing a polypeptide encoded by the nucleic acids of the invention, comprising culturing a recombinant host cell under conditions promoting expression of said polypeptide, wherein the recombinant host cell comprises at least one nucleic acid of the invention. A preferred process provided by the invention further comprises purifying said polypeptide. In another aspect of the invention, the polypeptide produced by said process is provided.

Further aspects of the invention are isolated antibodies that bind to the polypeptides of the invention, preferably monoclonal antibodies, also preferably humanized antibodies or humanized antibodies, and preferably wherein the antibody inhibits the activity of said polypeptides.

The invention additionally provides a method of designing an inhibitor of the polypeptides of the invention, the method comprising the steps of determining the three-dimensional structure of any such polypeptide, analyzing the three-dimensional structure for the likely binding sites of substrates, synthesizing a molecule that incorporates a predicted reactive site, and determining the polypeptide-inhibiting activity of the molecule.

In a further aspect of the invention, a method is provided for identifying compounds that alter IMX129840 cytokine polypeptide activity comprising
(a) mixing a test compound with a polypeptide of the invention; and
(b) determining whether the test compound alters the IMX129840 cytokine polypeptide activity of said polypeptide.

In another aspect of the invention, a method is provided identifying compounds that inhibit the binding activity of IMX129840 cytokine polypeptides comprising
(a) mixing a test compound with a polypeptide of the invention and a binding partner of said polypeptide; and
(b) determining whether the test compound inhibits the binding activity of said polypeptide.
   In preferred embodiments, the binding partner is a cell surface receptor that is a member of the immunoglobulin superfamily; more preferably, the binding partner is a member of the cytokine receptor family.

The invention also provides a method for increasing proliferation and/or differentiation of cells from pluripotent stem cell precursors, comprising providing at least one compound selected from the group consisting of the polypeptides of the invention and agonists of said polypeptides; with a preferred embodiment of the method further comprising increasing said activities in a patient by administering at least one polypeptide of the invention.

Further provided by the invention is a method for decreasing proliferation and/or differentiation of cells from pluripotent stem cell precursors, comprising providing at least one antagonist of the polypeptides of the invention; with a preferred embodiment of the method further comprising decreasing said activities in a patient by administering at least one antagonist of the polypeptides of the invention, and with a further preferred embodiment wherein the antagonist is an antibody that inhibits the activity of any of said polypeptides.

The invention additionally provides a method for increasing the number of cytokine-receptor-bearing cells or their developmentally committed progeny, through increased cell proliferation and/or altered cell differentiation, comprising contacting said cytokine-receptor-bearing cells with polypeptides of the invention or agonists thereof. In preferred embodiments, the cytokine-receptor-bearing cells are pluripotent cells, and in further preferred embodiments, the cytokine-receptor-bearing cells are cells of the hematopoietic system.

In other aspects of the invention, methods are provided for treating cytopenias for cytokine-receptor-bearing cells or their developmentally committed progeny, comprising administering to a patient a therapeutically effective amount of one or more polypeptides of the invention or agonists thereof. In preferred embodiments, the patient is a human patient; and in further preferred embodiments, the cytopenia is a disease affecting hematopoietic cells. Methods are also provided for treating the hypoproliferation of cytokine-receptor-bearing cells or their developmentally committed progeny, comprising administering to a patient a therapeutically effective amount of one or more antagonists of polypeptides of the invention. In preferred embodiments, the patient is a human patient; and in further preferred embodiments, the hypoproliferation is a cancerous or metastatic condition; and more preferably the hypoproliferation is a lymphoproliferation such as leukemia.

Also encompassed within the scope of the invention are methods for increasing immune activity against pathogens and/or tumors by increasing specific subclasses of immune cells with increased effector functions, comprising administering to a patient a therapeutically effective amount of one or more polypeptides of the invention or agonists thereof. In preferred embodiments, the patient is a human patient; and in a further preferred embodiment, the increased effector function is increased cytolytic lymphocyte function against virally infected or cancerous cells.

Further provided by the invention is a method for treating disorders of keratinocytes or other skin cells, for example disorders such as psoriasis, comprising providing at least one antagonist of the polypeptides of the invention; with an embodiment of the method further comprising treating a patient by administering at least one antagonist of the polypeptides of the invention, and with a further embodiment wherein the antagonist is an antibody that inhibits the activity of any of said polypeptides.

Other aspects of the invention are methods for treating disorders such as colitis, Crohn's disease, or other inflammatory bowel diseases, and methods for increasing epithelial barrier function in intestinal epithelia, such methods comprising providing at least one antagonist of the polypeptides of the invention; with an embodiment of the method further comprising treating a patient by administering at least one antagonist of the polypeptides of the invention, and with a further embodiment wherein the antagonist is an antibody that inhibits the activity of any of said polypeptides.

Also provided by the invention are methods for treating disorders such as asthma, allergy, or other inflammatory respiratory or lung diseases, and methods for increasing epithelial barrier function in lung epithelia, such methods comprising providing at least one compound selected from the group consisting of the polypeptides of the invention and agonists of said polypeptides; with a preferred embodiment of the method comprising treating a patient by administering at least one polypeptide of the invention.

### DETAILED DESCRIPTION OF THE INVENTION

Similarities of IMX129840 Cytokine Structure to Other 4AHB Cytokine Family Members

We have identified a group of structurally related 4AHB cytokines, clustered together within an approximately 120-kb region of human chromosome 19; these human cytokines have been named IMX129840-1, IMX129840-2, IMX129840-3, and IMX129840-4. The amino acid sequences of the IMX129840-1, -2, and -3 cytokine polypeptides are provided in SEQ ID NOs 2, 4, and 6, respectively, and an alignment showing the amino acid sequence similarities between these IMX129840 cytokines is presented in Table 1 in Example 1 below. We have also identified a partial amino acid sequence for IMX129840-4 cytokine polypeptide (SEQ ID NO:8); a partial amino acid sequence for the murine polypeptide corresponding to human IMX129840-1 (SEQ ID NO:10); and a complete amino acid sequence for the murine polypeptide corresponding to human IMX129840-2 (SEQ ID NO:12).

The typical structural elements common to members of the 4AHB cytokine polypeptide family include a signal sequence and four ‘core’ alpha helices. A signal sequence is also found at the N-termini of IMX129840 cytokine polypeptides, and is followed, in N-to-C order, by helix A, loop AB, helix B, loop BC, helix C, loop CD, and helix D. The IMX129840-1 cytokine polypeptide has a signal sequence beginning approximately between amino acid 7 through amino acid 9 and extending to approximately between amino acid 19 through amino acid 21 of SEQ ID NO:2, with the mature polypeptide produced by cleavage of the signal peptide (amino acids 1 through 19, 20, or 21 of SEQ ID NO:2) predicted to have an amino acid sequence beginning at amino acid 20, 21, or 22 of SEQ ID NO:2. Similarly, the murine IMX129840-1 cytokine polypeptide is predicted to have a signal sequence cleaved following amino acid 19 of SEQ ID NO:10 to produce a mature polypeptide with an N-terminal residue corresponding to amino acid 20 of SEQ ID NO:10. The IMX129840-2 and -3 cytokine polypeptides each have a signal sequence extending from approximately amino acid 13 to amino acid 25 of SEQ ID NOs 4 and 6, with the mature polypeptide produced by cleavage of the signal peptide (amino acids 1 through 25 of SEQ ID NOs 4 and 6) predicted in each case to have an amino acid sequence beginning at amino acid 26 of SEQ ID NOs 4 and 6. The murine IMX129840-2 cytokine polypeptide is predicted to have a signal sequence cleaved following amino acid 28 of SEQ ID NO:12 to produce a mature polypeptide with an N-terminal residue corresponding to amino acid 29 of SEQ ID NO:12.

The approximate locations of the four alpha helices in the human IMX129840-1, -2, and -3 cytokine polypeptide sequences (SEQ ID NOs 2, 4, and 6 respectively) and in the murine IMX129840-2 cytokine polypeptide (SEQ ID NO:12) are shown in the table below. The locations of these helices within IMX129840 cytokine polypeptides were determined by using the GeneFold program (described in more detail in Example 1 below) to find the regions in IMX129840-1 that fit most closely to the known alpha helices of cytokine template polypeptide structures such as GM-CSF and ovine interferon-tau, and the regions of murine IMX129840-2 that fit most closely to GM-CSF and a form of IL-3 (Protein Data Bank entry 1jli), and predicting the corresponding locations within IMX129840-2 and -3 based on their high degree of amino acid sequence similarity to IMX129840-1 (as shown in Table 1 of Example 1 below). Note that the overlap between the predicted extent of helix B and helix C results from the loop BC region between these helices assuming an extended conformation in some GeneFold template structures and a helical structure in others, consistent with the loop BC region being a flexible region that can have varied conformations in different 4AHB cytokines. Therefore, IMX129840 cytokine polypeptides have an overall structure consistent with that of other 4AHB cytokine polypeptides.

| | Location of Helix | | | | | | | | | | | |
|---|---|---|---|---|---|---|---|---|---|---|---|---|
| | Begins Between Amino | | | | | | Ends Between Amino | | | | | |
| | Acid A Through B | | | | | | Acid Y Through Z | | | | | |
| | SEQ ID | | SEQ ID NOs | | SEQ ID | | SEQ ID | | SEQ ID NOs | | SEQ ID | |
| | NO: 2 | | 4 and 6 | | NO: 12 | | NO: 2 | | 4 and 6 | | NO: 12 | |
| | A | B | A | B | A | B | Y | Z | Y | Z | Y | Z |
| Helix A | 44 | 47 | 51 | 54 | 55 | 63 | 59 | 61 | 66 | 68 | 73 | 85 |
| Helix B | 89 | 91 | 96 | 98 | 89 | 101 | 102 | 112 | 109 | 121 | 114 | 119 |
| Helix C | 111 | 116 | 120 | 125 | 124 | 130 | 131 | 135 | 140 | 144 | 137 | 146 |
| Helix D | 143 | 146 | 152 | 155 | 154 | 157 | 157 | 159 | 166 | 168 | 168 | 168 |

The skilled artisan will recognize that the boundaries of the regions of IMX129840 cytokine polypeptides described above are approximate and that the precise boundaries of such domains, as for example the boundaries of the signal sequence (which can be predicted by using computer programs available for that purpose), can also differ from member to member within the IMX129840 cytokine polypeptide family.

Biological Activities and Functions of IMX129840 Cytokine Polypeptides

PCR amplification from tissue-specific cDNA libraries was performed to detect IMX129840 cytokine cDNA sequences. The results of these experiments show that IMX129840-1 cytokine transcripts are found in several types of adult and fetal cells, including but not limited to adult skin, lung, kidney, pancreas, thymus, and leukocyte cells; fetal liver, brain, and thymus, and CX-1 colon adenocarcinoma cells.

Typical biological activities or functions associated with IMX129840 cytokine polypeptides are stimulation of the proliferation and/or differentiation of cells from pluripotent stem cell precursors. IMX129840 cytokine polypeptides are also associated with the formation and/or maintenance of barrier function in epithelia such as lung and intestinal epithelia. IMX129840 cytokine polypeptides having stimulation of cell proliferation activity bind receptor polypeptides. The receptor-binding activity is associated with domains comprising helix A and helix D of IMX129840 cytokine polypeptides. Thus, for uses requiring receptor-binding activity, preferred IMX129840 cytokine polypeptides include those having helix A and helix D and exhibiting stimulation of cell proliferation activity. Preferred IMX129840 cytokine polypeptides further include oligomers or fusion polypeptides comprising at least one alpha helix portion of one or more IMX129840 cytokine polypeptides, and fragments of any of these polypeptides that have stimulation of cell proliferation activity. The receptor-dependent stimulation of cell proliferation activity of IMX129840 cytokine polypeptides can be determined, for example, in a cell proliferation assay using BAF cells transfected with nucleic acid constructs directing the expression of receptor polypeptide chains (see, for example, FIG. 6 of Kallen et al., 1999, J Biol Chem 274: 11859-11867). Alternatively, the effect that treatment of cells with IMX129840 cytokine polypeptides has on activation of intracellular signaling pathways can be assayed by measuring the phosphorylation of receptor polypeptide chains or other targets of signaling pathway kinases such as targets of JAK family members (see, for example, FIG. 2 of Kallen et al., 1999, J Biol Chem 274: 11859-11867). IMX129840 cytokine polypeptides having stimulation of cell proliferation activity preferably have at least 10% (more preferably, at least 25%, and most preferably, at least 50%) of the maximal stimulation of cell proliferation activity of IL-6 as measured in FIG. 6A of Kallen et al., 1999, J Biol Chem 274: 11859-11867. IMX129840 cytokine polypeptides having stimulation of intracellular signalling activity preferably have at least 10% (more preferably, at least 25%, and most preferably, at least 50%) of the maximal phosphorylation of intracellular signaling pathway components activity of IL-6 as measured in FIG. 2A of Kallen et al., 1999, J Biol Chem 274: 11859-11867. The effect that IMX129840 cytokine polypeptides and agonists or antagonists thereof have on epithelial barrier function can be assayed by measurement of transepithelial electrical resistance (TER) as described in Ahdieh et al., 2001, Am J Physiol Cell Physiol 281: C2029-2038. IMX129840 cytokine polypeptides and agonists or antagonists that modify epithelial barrier function increase or decrease TER by at least 15% of the TER measured in untreated epithelia. The term “IMX129840 cytokine polypeptide activity,” as used herein, includes any one or more of the following: stimulation of cell proliferation activity and phosphorylation of intracellular signaling pathway components activity, modification of epithelial barrier function, and the ex vivo and in vivo activities of IMX129840 cytokine family polypeptides (for example, IMX129840-1, -2, -3, and/or -4). The degree to which individual members of the IMX129840 cytokine polypeptide family and fragments and other derivatives of these polypeptides exhibit these activities can be determined by standard assay methods, particularly assays such as those disclosed in Kallen et al., 1999, J Biol Chem 274: 11859-11867. Additional exemplary assays are disclosed herein; those of skill in the art will appreciate that other, similar types of assays can be used to measure IMX129840 cytokine family biological activities.

Another aspect of the biological activity of IMX129840 cytokine polypeptides is the ability of members of this polypeptide family to bind particular binding partners such as cell surface receptors that are members of the immunoglobulin superfamily, and more particularly to members of the cytokine receptor family. The term “binding partner,” as used herein, includes ligands, receptors, substrates, antibodies, other IMX129840 cytokine polypeptides, the same IMX129840 cytokine polypeptide (in the case of homotypic interactions or formation of multimers), and any other molecule that interacts with a IMX129840 cytokine polypeptide through contact or proximity between particular portions of the binding partner and the IMX129840 cytokine polypeptide. Because helix A and helix D of IMX129840 cytokine polypeptides are likely to be involved in the cytokine-receptor interaction, mutations of hydrophobic or charged residues within these helices are expected to alter the binding of IMX129840 cytokine polypeptides to receptor polypeptides; such mutations are likely to disrupt cytokine-receptor binding but may increase the strength of this interaction. By binding to one or more components of a cytokine receptor complex, or by binding to some components but not others, an altered IMX129840 cytokine polypeptide would likely prevent binding by the native IMX129840 cytokine polypeptide(s), and so act in a dominant negative fashion to inhibit the biological activities mediated via binding of IMX129840 cytokine polypeptides to cytokine receptors (see, for example, Tables I and II of interactions (Grötzinger et al., 1997, PROTEINS: Structure, Function, and Genetics 27: 96-109). Suitable assays to detect or measure the binding between IMX129840 cytokine polypeptides and their binding partners are well known to those of skill in the art and are described herein.

IMX129840 cytokine polypeptides are involved in diseases or conditions that share as a common feature proliferation and/or differentiation of cells from pluripotent stem cell precursors, or defects in such proliferative and/or developmental processes, in their etiology. Blocking or inhibiting the interactions between members of the IMX129840 cytokine polypeptide family and their substrates, ligands, receptors, binding partners, and or other interacting polypeptides is an aspect of the invention and provides methods for treating or ameliorating diseases and conditions involving excess proliferation and/or differentiation of cells from pluripotent stem cell precursors, through the use of inhibitors of IMX129840 cytokine polypeptide activity. Examples of such inhibitors or antagonists are described in more detail below. For conditions involving inadequate proliferation and/or differentiation of cells from pluripotent stem cell precursors, methods of treating or ameliorating these conditions comprise increasing the amount or activity of IMX129840 cytokine polypeptides by providing isolated IMX129840 cytokine polypeptides or active fragments or fusion polypeptides thereof, or by providing compounds (agonists) that activate endogenous or exogenous IMX129840 cytokine polypeptides. Additional uses for IMX129840 cytokine polypeptides include diagnostic reagents for conditions and diseases involving the proliferation or the development of cells from pluripotent stem cell precursors, research reagents for investigation of proliferation and/or differentiation of cells from pluripotent stem cell precursors, or as a carrier/targeting polypeptide to deliver therapeutic agents to cells expressing IMX129840 cytokine receptors.

IMX129840 Cytokine Polypeptides

A IMX129840 cytokine polypeptide is a polypeptide that shares a sufficient degree of amino acid identity or similarity to an IMX129840 cytokine polypeptide of SEQ ID NOs 2, 4, or 6 to (A) be identified by those of skill in the art as a polypeptide likely to share particular structural domains and/or (B) have biological activities in common with the IMX129840 cytokine polypeptide of SEQ ID NOs 2, 4, or 6 and/or (C) bind to antibodies that also specifically bind to other IMX129840 cytokine polypeptides. IMX129840 cytokine polypeptides can be isolated from naturally occurring sources, or have the same structure as naturally occurring IMX129840 cytokine polypeptides, or can be produced to have structures that differ from naturally occurring IMX129840 cytokine polypeptides. Polypeptides derived from any IMX129840 cytokine polypeptide by any type of alteration (for example, but not limited to, insertions, deletions, or substitutions of amino acids; changes in the state of glycosylation of the polypeptide; refolding or isomerization to change its three-dimensional structure or self-association state; and changes to its association with other polypeptides or molecules) are also IMX129840 cytokine polypeptides. Therefore, the polypeptides provided by the invention include polypeptides characterized by amino acid sequences similar to those of the IMX129840 cytokine polypeptides described herein, but into which modifications are naturally provided or deliberately engineered. A polypeptide that shares biological activities in common with IMX129840 cytokine polypeptides is a polypeptide having IMX129840 cytokine polypeptide activity. Examples of biological activities exhibited by IMX129840 cytokine polypeptides include, without limitation, stimulation of proliferation and/or differentiation of cells from pluripotent stem cell precursors.

The present invention provides both full-length and mature forms of IMX129840 cytokine polypeptides. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides can be encoded by a single genetic locus if multiple mRNA forms are produced from that locus by alternative splicing or by the use of multiple translation initiation sites. The “mature form” of a polypeptide refers to a polypeptide that has undergone post-translational processing steps such as cleavage of the signal sequence or proteolytic cleavage to remove a prodomain. Multiple mature forms of a particular full-length polypeptide may be produced, for example by cleavage of the signal sequence at multiple sites, or by differential regulation of proteases that cleave the polypeptide. The mature form(s) of such polypeptide can be obtained by expression, in a suitable mammalian cell or other host cell, of a nucleic acid molecule that encodes the full-length polypeptide. The sequence of the mature form of the polypeptide may also be determinable from the amino acid sequence of the full-length form, through identification of signal sequences or protease cleavage sites. The IMX129840 cytokine polypeptides of the invention also include those that result from post-transcriptional or post-translational processing events such as alternate mRNA processing which can yield a truncated but biologically active polypeptide, for example, a naturally occurring soluble form of the polypeptide. Also encompassed within the invention are variations attributable to proteolysis such as differences in the N- or C-termini upon expression in different types of host cells, due to proteolytic removal of one or more terminal amino acids from the polypeptide (generally from 1-5 terminal amino acids).

The invention further includes IMX129840 cytokine polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the invention in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase. In general, glycosylated polypeptides of the invention can be incubated with a molar excess of glycopeptidase (Boehringer Mannheim).

Species homologues of IMX129840 cytokine polypeptides and of nucleic acids encoding them are also provided by the present invention. As used herein, a “species homologue” is a polypeptide or nucleic acid with a different species of origin from that of a given polypeptide or nucleic acid, but with significant sequence similarity to the given polypeptide or nucleic acid, as determined by those of skill in the art. Species homologues can be isolated and identified by making suitable probes or primers from polynucleotides encoding the amino acid sequences provided herein and screening a suitable nucleic acid source from the desired species. The invention also encompasses allelic variants of IMX129840 cytokine polypeptides and nucleic acids encoding them; that is, naturally-occurring alternative forms of such polypeptides and nucleic acids in which differences in amino acid or nucleotide sequence are attributable to genetic polymorphism (allelic variation among individuals within a population).

Fragments of the IMX129840 cytokine polypeptides of the present invention are encompassed by the present invention and can be in linear form or cyclized using known methods, for example, as described in Saragovi et al., Bio/Technology 10, 773-778 (1992) and in McDowell et al., J. Amer. Chem. Soc. 114 9245-9253 (1992). Polypeptides and polypeptide fragments of the present invention, and nucleic acids encoding them, include polypeptides and nucleic acids with amino acid or nucleotide sequence lengths that are at least 25% (more preferably at least 50%, or at least 60%, or at least 70%, and most preferably at least 80%) of the length of a IMX129840 cytokine polypeptide and have at least 60% sequence identity (more preferably at least 70%, at least 75%, at least 80%, at least 85%, at least 90%, at least 95%, at least 97.5%, or at least 99%, and most preferably at least 99.5%) with that IMX129840 cytokine polypeptide or encoding nucleic acid, where sequence identity is determined by comparing the amino acid sequences of the polypeptides when aligned so as to maximize overlap and identity while minimizing sequence gaps. Also included in the present invention are polypeptides and polypeptide fragments, and nucleic acids encoding them, that contain or encode a segment preferably comprising at least 8, or at least 10, or preferably at least 15, or more preferably at least 20, or still more preferably at least 30, or most preferably at least 40 contiguous amino acids. Such polypeptides and polypeptide fragments may also contain a segment that shares at least 70% sequence identity (more preferably at least 70%, at least 75%, at least 80%, at least 85%, at least 90%, at least 95%, at least 97.5%, or at least 99%, and most preferably at least 99.5%) with any such segment of any IMX129840 cytokine polypeptide, where sequence identity is determined by comparing the amino acid sequences of the polypeptides when aligned so as to maximize overlap and identity while minimizing sequence gaps. The percent identity of two amino acid or two nucleic acid sequences can be determined by visual inspection and mathematical calculation, or more preferably, the comparison is done by comparing sequence information using a computer program. An exemplary, preferred computer program is the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.0.9, available for use via the National Library of Medicine website ncbi.nlm.nih.gov/gorf/wblast2.cgi, or the UW-BLAST 2.0 algorithm. Standard default parameter settings for UW-BLAST 2.0 are described at the following Internet site: sapiens.wustl.edu/blast/blast/#Features. In addition, the BLAST algorithm uses the BLOSUM62 amino acid scoring matix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

The present invention also provides for soluble forms of IMX129840 cytokine polypeptides comprising certain fragments or domains of these polypeptides. Soluble polypeptides are polypeptides that are capable of being secreted from the cells in which they are expressed. A secreted soluble polypeptide can be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble forms of IMX129840 cytokine polypeptides is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures.

“An isolated polypeptide consisting essentially of an amino acid sequence” means that the polypeptide may have, in addition to said amino acid sequence, additional material covalently linked to either or both ends of the polypeptide, said additional material preferably between 1 and 10,000 additional amino acids covalently linked to either end, each end, or both ends of polypeptide, and more preferably between 1 and 1,000 additional amino acids covalently linked to either end, each end, or both ends of the polypeptide, and most preferably between 1 and 100 additional amino acids covalently linked to either end, each end, or both ends of the polypeptide. In preferred embodiments, covalent linkage of additional amino acids to either end, each end, or both ends of the polypeptide results in a novel combined amino acid sequence that is neither naturally occurring nor disclosed in the art.

In another aspect of the invention, preferred polypeptides comprise various combinations of IMX129840 cytokine polypeptide structures, such as helices A, B, C, and D and the inter-helix loops AB, BC, and CD. Accordingly, polypeptides of the present invention and nucleic acids encoding them include those comprising or encoding two or more copies of helix A, two or more copies of helix D, or at least one copy of each. A further embodiment of the invention is an isolated IMX129840 cytokine polypeptide consisting of the following, in N-to-C order: a polypeptide consisting essentially of helix A, covalently linked to a polypeptide consisting essentially of helix B, covalently linked to a polypeptide consisting essentially of helix C, covalently linked to a polypeptide consisting essentially of helix D, wherein a polypeptide consisting essentially of a given helix of the IMX129840 cytokine polypeptide may include a naturally occurring or a modified inter-helix loop amino acid sequence, for example, an inter-helix loop sequence in which conservative substitutions have been made of one or more amino acids.

Further modifications in the peptide or DNA sequences can be made by those skilled in the art using known techniques. Modifications of interest in the polypeptide sequences can include the alteration, substitution, replacement, insertion or deletion of a selected amino acid. For example, one or more of the cysteine residues can be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the polypeptide can be modified to preclude glycosylation, allowing expression of a reduced carbohydrate analog in mammalian and yeast expression systems. N-glycosylation sites in eukaryotic polypeptides are characterized by an amino acid triplet Asn-X—Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in polypeptides include those described in U.S. Pat. No. 5,071,972 and EP 276,846. Additional variants within the scope of the invention include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide or a substantial equivalent thereof. One example is a variant that binds with essentially the same binding affinity as does the native form. Binding affinity can be measured by conventional procedures, e.g., as described in U.S. Pat. No. 5,512,457 and as set forth herein.

Other derivatives include covalent or aggregative conjugates of the polypeptides with other polypeptides or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Encompassed by the invention are oligomers or fusion polypeptides that contain a IMX129840 cytokine polypeptide, one or more fragments of IMX129840 cytokine polypeptides, or any of the derivative or variant forms of IMX129840 cytokine polypeptides as disclosed herein. In particular embodiments, the oligomers comprise soluble IMX129840 cytokine polypeptides. Oligomers can be in the form of covalently linked or non-covalently-linked multimers, including dimers, trimers, or higher oligomers. In one aspect of the invention, the oligomers maintain the binding ability of the polypeptide components and provide therefor, bivalent, trivalent, etc., binding sites. In an alternative embodiment the invention is directed to oligomers comprising multiple IMX129840 cytokine polypeptides joined via covalent or non-covalent interactions between peptide moieties fused to the polypeptides, such peptides having the property of promoting oligomerization. Leucine zippers and certain polypeptides derived from antibodies are among the peptides that can promote oligomerization of the polypeptides attached thereto, as described in more detail below.

In embodiments where variants of the IMX129840 cytokine polypeptides are constructed to include a membrane-spanning domain, they will form a Type I membrane polypeptide. Membrane-spanning IMX129840 cytokine polypeptides can be fused with extracellular domains of receptor polypeptides for which the ligand is known. Such fusion polypeptides can then be manipulated to control the intracellular signaling pathways triggered by the membrane-spanning IMX129840 cytokine polypeptide. IMX129840 cytokine polypeptides that span the cell membrane can also be fused with agonists or antagonists of cell-surface receptors, or cellular adhesion molecules to further modulate IMX129840 cytokine intracellular effects. In another aspect of the present invention, other interleukin or cytokine polypeptides can be situated between the preferred IMX129840 cytokine polypeptide fragment and other fusion polypeptide domains.

Immunoglobulin-based Oligomers. The polypeptides of the invention or fragments thereof can be fused to molecules such as immunoglobulins for many purposes, including increasing the valency of polypeptide binding sites. For example, fragments of a IMX129840 cytokine polypeptide can be fused directly or through linker sequences to the Fc portion of an immunoglobulin. For a bivalent form of the polypeptide, such a fusion could be to the Fc portion of an IgG molecule. Other immunoglobulin isotypes can also be used to generate such fusions. For example, a polypeptide-IgM fusion would generate a decavalent form of the polypeptide of the invention. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred Fc polypeptides comprise an Fc polypeptide derived from a human IgG1 antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Polypeptides”, in Current Protocols in Immunology, Suppl. 4, pages 10.19.1-10.19.11, 1992). Methods for preparation and use of immunoglobulin-based oligomers are well known in the art. One embodiment of the present invention is directed to a dimer comprising two fusion polypeptides created by fusing a polypeptide of the invention to an Fc polypeptide derived from an antibody. A gene fusion encoding the polypeptide/Fc fusion polypeptide is inserted into an appropriate expression vector. Polypeptide/Fc fusion polypeptides are expressed in host cells transformed with the recombinant expression vector, and allowed to assemble much like antibody molecules, whereupon interchain disulfide bonds form between the Fc moieties to yield divalent molecules. One suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four IMX129840 cytokine extracellular regions.

Peptide-linker Based Oligomers. Alternatively, the oligomer is a fusion polypeptide comprising multiple IMX129840 cytokine polypeptides, with or without peptide linkers (spacer peptides). Among the suitable peptide linkers are those described in U.S. Pat. Nos. 4,751,180 and 4,935,233. A DNA sequence encoding a desired peptide linker can be inserted between, and in the same reading frame as, the DNA sequences of the invention, using any suitable conventional technique. For example, a chemically synthesized oligonucleotide encoding the linker can be ligated between the sequences. In particular embodiments, a fusion polypeptide comprises from two to four soluble IMX129840 cytokine polypeptides, separated by peptide linkers. Suitable peptide linkers, their combination with other polypeptides, and their use are well known by those skilled in the art.

Leucine-Zippers. Another method for preparing the oligomers of the invention involves use of a leucine zipper. Leucine zipper domains are peptides that promote oligomerization of the polypeptides in which they are found. Leucine zippers were originally identified in several DNA-binding polypeptides (Landschulz et al., Science 240:1759, 1988), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art.

Other fragments and derivatives of the sequences of polypeptides which would be expected to retain polypeptide activity in whole or in part and may thus be useful for screening or other immunological methodologies can also be made by those skilled in the art given the disclosures herein. Such modifications are believed to be encompassed by the present invention.

Nucleic Acids Encoding IMX129840 Cytokine Polypeptides

Encompassed within the invention are nucleic acids encoding IMX129840 cytokine polypeptides. These nucleic acids can be identified in several ways, including isolation of genomic or cDNA molecules from a suitable source. Nucleotide sequences corresponding to the amino acid sequences described herein, to be used as probes or primers for the isolation of nucleic acids or as query sequences for database searches, can be obtained by “back-translation” from the amino acid sequences, or by identification of regions of amino acid identity with polypeptides for which the coding DNA sequence has been identified. The well-known polymerase chain reaction (PCR) procedure can be employed to isolate and amplify a DNA sequence encoding a IMX129840 cytokine polypeptide or a desired combination of IMX129840 cytokine polypeptide fragments. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990).

Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention are preferentially derived from human sources, but the invention includes those derived from non-human species, as well.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning. A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

“An isolated nucleic acid consisting essentially of a nucleotide sequence” means that the nucleic acid may have, in addition to said nucleotide sequence, additional material covalently linked to either or both ends of the nucleic acid molecule, said additional material preferably between 1 and 100,000 additional nucleotides covalently linked to either end, each end, or both ends of the nucleic acid molecule, and more preferably between 1 and 1,000 additional nucleotides covalently linked to either end, each end, or both ends of the nucleic acid molecule, and most preferably between 10 and 100 additional nucleotides covalently linked to either end, each end, or both ends of the nucleic acid molecule. In preferred embodiments, covalent linkage of additional nucleotides to either end, each end, or both ends of the nucleic acid molecule results in a novel combined nucleotide sequence that is neither naturally occurring nor disclosed in the art. An isolated nucleic acid consisting essentially of a nucleotide sequence may be an expression vector or other construct comprising said nucleotide sequence.

The present invention also includes nucleic acids that hybridize under moderately stringent conditions, and more preferably highly stringent conditions, to nucleic acids encoding IMX129840 cytokine polypeptides described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55 degrees C. (or other similar hybridization solutions, such as one containing about 50% formamide, with a hybridization temperature of about 42 degrees C.), and washing conditions of about 60 degrees C., in 0.5×SSC, 0.1% SDS. Generally, highly stringent conditions are defined as hybridization conditions as above, but with washing at approximately 68 degrees C., 0.2×SSC, 0.1% SDS. SSPE (1×SSPE is 0.15 M NaCl, 10 mM NaH.sub.2 PO.sub.4, and 1.25 mM EDTA, pH 7.4) can be substituted for SSC (1×SSC is 0.15 M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10.degrees C. less than the melting temperature (Tm) of the hybrid, where Tm is determined according to the following equations. For hybrids less than 18 base pairs in length, Tm (degrees C.)=2(# of A+T bases)+4(# of #G+C bases). For hybrids above 18 base pairs in length, Tm (degrees C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). Preferably, each such hybridizing nucleic acid has a length that is at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 85%, at least 90%, at least 95%, at least 97.5%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

The present invention also provides genes corresponding to the nucleic acid sequences disclosed herein. “Corresponding genes” or “corresponding genomic nucleic acids” are the regions of the genome that are transcribed to produce the mRNAs from which cDNA nucleic acid sequences are derived and can include contiguous regions of the genome necessary for the regulated expression of such genes. Corresponding genes can therefore include but are not limited to coding sequences, 5′ and 3′ untranslated regions, alternatively spliced exons, introns, promoters, enhancers, and silencer or suppressor elements. Corresponding genomic nucleic acids can include 10000 basepairs (more preferably, 5000 basepairs, still more preferably, 2500 basepairs, and most preferably, 1000 basepairs) of genomic nucleic acid sequence upstream of the first nucleotide of the genomic sequence corresponding to the initiation codon of the IMX129840 cytokine coding sequence, and 10000 basepairs (more preferably, 5000 basepairs, still more preferably, 2500 basepairs, and most preferably, 1000 basepairs) of genomic nucleic acid sequence downstream of the last nucleotide of the genomic sequence corresponding to the termination codon of the IMX129840 cytokine coding sequence. The corresponding genes or genomic nucleic acids can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” or “an isolated genomic nucleic acid” is a genomic nucleic acid that has been separated from the adjacent genomic sequences present in the genome of the organism from which the genomic nucleic acid was isolated.

Methods for Making and Purifying IMX129840 Cytokine Polypeptides

Methods for making IMX129840 cytokine polypeptides are described below. Expression, isolation, and purification of the polypeptides and fragments of the invention can be accomplished by any suitable technique, including but not limited to the following methods. The isolated nucleic acid of the invention can be operably linked to an expression control sequence such as the pDC409 vector (Giri et al., 1990, EMBO J., 13: 2821) or the derivative pDC412 vector (Wiley et al., 1995, Immunity 3: 673). The pDC400 series vectors are useful for transient mammalian expression systems, such as CV-1 or 293 cells. Alternatively, the isolated nucleic acid of the invention can be linked to expression vectors such as pDC312, pDC316, or pDC317 vectors. The pDC300 series vectors all contain the SV40 origin of replication, the CMV promoter, the adenovirus tripartite leader, and the SV40 polyA and termination signals, and are useful for stable mammalian expression systems, such as CHO cells or their derivatives. Other expression control sequences and cloning technologies can also be used to produce the polypeptide recombinantly, such as the pMT2 or pED expression vectors (Kaufman et al., 1991, Nucleic Acids Res. 19: 4485-4490; and Pouwels et al., 1985, Cloning Vectors: A Laboratory Manual, Elsevier, New York) and the GATEWAY Vectors (lifetech.com/Content/Tech-Online/molecular_biology/manuals_pps/11797016.pdf; Life Technologies; Rockville, Md.). In the GATEWAY system the isolated nucleic acid of the invention, flanked by attB sequences, can be recombined through an integrase reaction with a GATEWAY vector such as pDONR201 containing attP sequences. This provides an entry vector for the GATEWAY system containing the isolated nucleic acid of the invention. This entry vector can be further recombined with other suitably prepared expression control sequences, such as those of the pDC400 and pDC300 series described above. Many suitable expression control sequences are known in the art. General methods of expressing recombinant polypeptides are also described in R. Kaufman, Methods in Enzymology 185, 537-566 (1990). As used herein “operably linked” means that the nucleic acid of the invention and an expression control sequence are situated within a construct, vector, or cell in such a way that the polypeptide encoded by the nucleic acid is expressed when appropriate molecules (such as polymerases) are present. As one embodiment of the invention, at least one expression control sequence is operably linked to the nucleic acid of the invention in a recombinant host cell or progeny thereof, the nucleic acid and/or expression control sequence having been introduced into the host cell by transformation or transfection, for example, or by any other suitable method. As another embodiment of the invention, at least one expression control sequence is integrated into the genome of a recombinant host cell such that it is operably linked to a nucleic acid sequence encoding a polypeptide of the invention. In a further embodiment of the invention, at least one expression control sequence is operably linked to a nucleic acid of the invention through the action of a trans-acting factor such as a transcription factor, either in vitro or in a recombinant host cell.

In addition, a sequence encoding an appropriate signal peptide (native or heterologous) can be incorporated into expression vectors. The choice of signal peptide or leader can depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal peptide described in EP 367,566; the type I interleukin-1 receptor signal peptide described in U.S. Pat. No. 4,968,607; and the type II interleukin-1 receptor signal peptide described in EP 460,846. A DNA sequence for a signal peptide (secretory leader) can be fused in frame to the nucleic acid sequence of the invention so that the DNA is initially transcribed, and the mRNA translated, into a fusion polypeptide comprising the signal peptide. A signal peptide that is functional in the intended host cells is one that promotes insertion of the polypeptide into cell membranes, and most preferably, promotes extracellular secretion of the polypeptide from that host cell. The signal peptide is preferably cleaved from the polypeptide upon membrane insertion or secretion of polypeptide from the cell. The skilled artisan will also recognize that the position(s) at which the signal peptide is cleaved can differ from that predicted by computer program, and can vary according to such factors as the type of host cells employed in expressing a recombinant polypeptide. A polypeptide preparation can include a mixture of polypeptide molecules having different N-terminal amino acids, resulting from cleavage of the signal peptide at more than one site.

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning. A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable strain for DHFR selection is CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Alternatively, IMX129840 cytokine gene products can be obtained via homologous recombination, or “gene targeting,” techniques. Such techniques employ the introduction of exogenous transcription control elements (such as the CMV promoter or the like) in a particular predetermined site on the genome, to induce expression of the endogenous nucleic acid sequence of interest (see, for example, U.S. Pat. No. 5,272,071). The location of integration into a host chromosome or genome can be easily determined by one of skill in the art, given the known location and sequence of the gene. In a preferred embodiment, the present invention also contemplates the introduction of exogenous transcriptional control elements in conjunction with an amplifiable gene, to produce increased amounts of the gene product, again, without the need for isolation of the gene sequence itself from the host cell.

A number of types of cells can act as suitable host cells for expression of the polypeptide. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells or their derivatives such as Veggie CHO and related cell lines which grow in serum-free media (Rasmussen et al., 1998, Cytotechnology 28: 31), HeLa cells, BHK (ATCC CRL 10) cell lines, the CV1/EBNA cell line derived from the African green monkey kidney cell line CV1 (ATCC CCL 70) (McMahan et al., 1991, EMBO J. 10: 2821, 1991), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays. Alternatively, it is possible to produce the polypeptide in lower eukaryotes such as yeast or in prokaryotes such as bacteria. Suitable yeasts include Saccharomyces cerevisiae, Schizosaccharomyces pombe, Kluyveromyces strains, Candida, or any yeast strain capable of expressing heterologous polypeptides. Suitable bacterial strains include Escherichia coli, Bacillus subtilis, Salmonella typhimurium, or any bacterial strain capable of expressing heterologous polypeptides. If the polypeptide is made in yeast or bacteria, it may be desirable to modify the polypeptide produced therein, for example by phosphorylation or glycosylation of the appropriate sites, in order to obtain the functional polypeptide. Such covalent attachments can be accomplished using known chemical or enzymatic methods. The polypeptide can also be produced by operably linking the isolated nucleic acid of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988). Cell-free translation systems could also be employed to produce polypeptides using RNAs derived from nucleic acid constructs disclosed herein. A host cell that comprises an isolated nucleic acid of the invention, preferably operably linked to at least one expression control sequence, is a “recombinant host cell”.

The polypeptide of the invention can be prepared by culturing transformed host cells under culture conditions suitable to express the recombinant polypeptide. The resulting expressed polypeptide can then be purified from such culture (i.e., from culture medium or cell extracts) using known purification processes, such as selective precipitation with various salts, gel filtration, and ion exchange chromatography. The purification of the polypeptide can also include an affinity column containing agents which will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography using an antibody that specifically binds one or more IMX129840 cytokine epitopes. Alternatively, the polypeptide of the invention can also be expressed in a form which will facilitate purification. For example, it can be expressed as a fusion polypeptide, that is, it may be fused with maltose binding polypeptide (MBP), glutathione-5-transferase (GST8), thioredoxin (TRX), a polyHis peptide, and/or fragments thereof. Kits for expression and purification of such fusion polypeptides are commercially available from New England BioLabs (Beverly, Mass.), Pharmacia (Piscataway, N.J.) and InVitrogen, respectively. The polypeptide can also be tagged with an epitope and subsequently purified by using a specific antibody directed to such epitope. One such epitope (FLAG®) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous isolated recombinant polypeptide. The polypeptide thus purified is substantially free of other mammalian polypeptides and is defined in accordance with the present invention as an “isolated polypeptide”; such isolated polypeptides of the invention include isolated antibodies that bind to IMX129840 cytokine polypeptides, fragments, variants, binding partners etc. The polypeptide of the invention can also be expressed as a product of transgenic animals, e.g., as a component of the milk of transgenic cows, goats, pigs, or sheep which are characterized by somatic or germ cells containing a nucleotide sequence encoding the polypeptide.

It is also possible to utilize an affinity column comprising a polypeptide-binding polypeptide of the invention, such as a monoclonal antibody generated against polypeptides of the invention, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention. In this aspect of the invention, polypeptide-binding polypeptides, such as the anti-polypeptide antibodies of the invention or other polypeptides that can interact with the polypeptide of the invention, can be bound to a solid phase support such as a column chromatography matrix or a similar substrate suitable for identifying, separating, or purifying cells that express polypeptides of the invention on their surface. Adherence of polypeptide-binding polypeptides of the invention to a solid phase contacting surface can be accomplished by any means, for example, magnetic microspheres can be coated with these polypeptide-binding polypeptides and held in the incubation vessel through a magnetic field. Suspensions of cell mixtures are contacted with the solid phase that has such polypeptide-binding polypeptides thereon. Cells having polypeptides of the invention on their surface bind to the fixed polypeptide-binding polypeptide and unbound cells then are washed away. This affinity-binding method is useful for purifying, screening, or separating such polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner. Alternatively, mixtures of cells suspected of containing polypeptide-expressing cells of the invention first can be incubated with a biotinylated polypeptide-binding polypeptide of the invention. The resulting mixture then is passed through a column packed with avidin-coated beads, whereby the high affinity of biotin for avidin provides the binding of the polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

The polypeptide can also be produced by known conventional chemical synthesis. Methods for constructing the polypeptides of the present invention by synthetic means are known to those skilled in the art. The synthetically-constructed polypeptide sequences, by virtue of sharing primary, secondary or tertiary structural and/or conformational characteristics with IMX129840 cytokine polypeptides can possess biological properties in common therewith, including IMX129840 cytokine polypeptide activity. Thus, they can be employed as biologically active or immunological substitutes for natural, purified polypeptides in screening of therapeutic compounds and in immunological processes for the development of antibodies.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Antagonists and Agonists of IMX129840 Cytokine Polypeptides

Any method which neutralizes IMX129840 cytokine polypeptides or inhibits expression of the IMX129840 cytokine genes (either transcription or translation) can be used to reduce the biological activities of IMX129840 cytokine polypeptides. In particular embodiments, antagonists inhibit the binding of at least one IMX129840 cytokine polypeptide to cells, thereby inhibiting biological activities induced by the binding of those IMX129840 cytokine polypeptides to the cells. In certain other embodiments of the invention, antagonists can be designed to reduce the level of endogenous IMX129840 cytokine gene expression, e.g., using well-known antisense or ribozyme approaches to inhibit or prevent translation of IMX129840 cytokine mRNA transcripts; triple helix approaches to inhibit transcription of IMX129840 cytokine family genes; or targeted homologous recombination to inactivate or “knock out” the IMX129840 cytokine genes or their endogenous promoters or enhancer elements. Antisense, ribozyme, double-stranded (ds) RNA for RNAi methods, and triple helix antagonists, examples of nucleic acid antagonists, can be designed to reduce or inhibit either unimpaired, or if appropriate, mutant IMX129840 cytokine gene activity. Techniques for the production and use of such molecules are well known to those of skill in the art.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to a IMX129840 cytokine mRNA. The antisense oligonucleotides will bind to the complementary target gene mRNA transcripts and prevent translation. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). In the case of double-stranded antisense nucleic acids, a single strand of the duplex DNA can thus be tested, or triplex formation can be assayed. The ability to hybridize will depend on both the degree of complementarity and the length of the antisense nucleic acid. Preferred oligonucleotides are complementary to the 5′ end of the message, e.g., the 5′ untranslated sequence up to and including the AUG initiation codon. However, oligonucleotides complementary to the 5′- or 3′-non- translated, non-coding regions of the IMX129840 cytokine gene transcript, or to the coding regions, could be used in an antisense approach to inhibit translation of endogenous IMX129840 cytokine mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleosides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). Oligonucleotides of the first type are also known in the art as “gapmers” or gapped oligonucleotides. Oligonucleotides of the second type are also known in the art as “hemimers” or “wingmers”. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., 1989, Proc Natl Acad Sci U.S.A. 86: 6553-6556; Lemaitre et al., 1987, Proc Natl Acad Sci 84: 648-652; PCT Publication No. WO88/09810), or hybridization-triggered cleavage agents or intercalating agents. (See, e.g., Zon, 1988, Pharm. Res. 5: 539-549). The antisense molecules should be delivered to cells which express the IMX129840 cytokine transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous IMX129840 cytokine gene transcripts and thereby prevent translation of the IMX129840 cytokine mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave GRK7 mRNA transcripts can also be used to prevent translation of GRK7 mRNA and expression of GRK7 polypeptides. (See, e.g., PCT International Publication WO90/11364 and U.S. Pat. No. 5,824,519). The ribozymes that can be used in the present invention include hairpin ribozymes (U.S. Pat. No. 6,221,661), hammerhead ribozymes (Haseloff and Gerlach, 1988, Nature, 334:585-591), RNA endoribonucleases (International Patent Application No. WO 88/04300; Been and Cech, 1986, Cell, 47:207-216). As in the antisense approach, the ribozymes can be composed of modified oligonucleotides (e.g. for improved stability, targeting, etc.) and should be delivered to cells which express the IMX129840 cytokine polypeptide in vivo. A preferred method of delivery involves using a DNA construct “encoding” the ribozyrne under the control of a strong constitutive pol III or pol II promoter, so that transfected cells will produce sufficient quantities of the ribozyme to destroy endogenous IMX129840 cytokine messages and inhibit translation. Because ribozymes, unlike antisense molecules, are catalytic, a lower intracellular concentration is required for efficiency.

Alternatively, endogenous IMX129840 cytokine gene expression can be reduced by targeting deoxyribonucleotide sequences complementary to the regulatory region of the target gene (i.e., the target gene promoter and/or enhancers) to form triple helical structures that prevent transcription of the target IMX129840 cytokine gene. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. U.S.A. 85:7448-7451). Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok, Tabara, and Mello, 2000, Genetic requirements for inheritance of RNAi in C. elegans, Science 287 (5462): 2494-2497), or the introduction of transgenes (Dernburg et al., 2000, Transgene-mediated cosuppression in the C. elegans germ line, Genes Dev. 14 (13): 1578-1583) are used to inhibit the expression of specific target genes. However this approach can be adapted for use in humans provided the recombinant DNA constructs are directly administered or targeted to the required site in vivo using appropriate vectors such as viral vectors.

Organisms that have enhanced, reduced, or modified expression of the gene(s) corresponding to the nucleic acid sequences disclosed herein are provided. The desired change in gene expression can be achieved through the use of antisense nucleic acids or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39). Transgenic animals that have multiple copies of the gene(s) corresponding to the nucleic acid sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1). In addition, organisms are provided in which the gene(s) corresponding to the nucleic acid sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523). These organisms with altered gene expression are preferably eukaryotes and more preferably are mammals. Such organisms are useful for the development of non-human models for the study of disorders involving the corresponding gene(s), and for the development of assay systems for the identification of molecules that interact with the polypeptide product(s) of the corresponding gene(s).

Also encompassed within the invention are IMX129840 cytokine polypeptide variants with partner binding sites that have been altered in conformation so that (1) the IMX129840 cytokine variant will still bind to its partner(s), but a specified small molecule will fit into the altered binding site and block that interaction, or (2) the IMX129840 cytokine variant will no longer bind to its partner(s) unless a specified small molecule is present (see for example Bishop et al., 2000, Nature 407: 395-401). Nucleic acids encoding such altered IMX129840 cytokine polypeptides can be introduced into organisms according to methods described herein, and can replace the endogenous nucleic acid sequences encoding the corresponding IMX129840 cytokine polypeptide. Such methods allow for the interaction of a particular IMX129840 cytokine polypeptide with its binding partners to be regulated by administration of a small molecule compound to an organism, either systemically or in a localized manner.

The IMX129840 cytokine polypeptides themselves can also be employed in inhibiting a biological activity of IMX129840 cytokine in in vitro or in vivo procedures. Encompassed within the invention are mutated regions of IMX129840 cytokine polypeptides that act as “dominant negative” inhibitors of native IMX129840 cytokine polypeptide function when expressed as fragments or as components of fusion polypeptides. For example, an altered polypeptide region of the present invention can be used to inhibit binding of IMX129840 cytokine polypeptides to endogenous binding partners. Such use effectively would block IMX129840 cytokine polypeptide interactions and inhibit IMX129840 cytokine polypeptide activities. Furthermore, antibodies which bind to IMX129840 cytokine polypeptides often inhibit IMX129840 cytokine polypeptide activity and act as antagonists. For example, antibodies that specifically recognize one or more epitopes of IMX129840 cytokine polypeptides, or epitopes of conserved variants of IMX129840 cytokine polypeptides, or peptide fragments of the IMX129840 cytokine polypeptide can be used in the invention to inhibit IMX129840 cytokine polypeptide activity. Such antibodies include but are not limited to polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Alternatively, purified and modified IMX129840 cytokine polypeptides of the present invention can be administered to modulate interactions between IMX129840 cytokine polypeptides and IMX129840 cytokine binding partners that are not membrane-bound. Such an approach will allow an alternative method for the modification of IMX129840 cytokine-influenced bioactivity.

In an alternative aspect, the invention further encompasses the use of agonists of IMX129840 cytokine polypeptide activity to treat or ameliorate the symptoms of a disease for which increased IMX129840 cytokine polypeptide activity is beneficial. In a preferred aspect, the invention entails administering compositions comprising an IMX129840 cytokine nucleic acid or an IMX129840 cytokine polypeptide to cells in vitro, to cells ex vivo, to cells in vivo, and/or to a multicellular organism such as a vertebrate or mammal. Preferred therapeutic forms of IMX129840 cytokine are soluble forms, as described above. In still another aspect of the invention, the compositions comprise administering a IMX129840 cytokine-encoding nucleic acid for expression of a IMX129840 cytokine polypeptide in a host organism for treatment of disease. Particularly preferred in this regard is expression in a human patient for treatment of a dysfunction associated with aberrant (e.g., decreased) endogenous activity of a IMX129840 cytokine family polypeptide. Furthermore, the invention encompasses the administration to cells and/or organisms of compounds found to increase the endogenous activity of IMX129840 cytokine polypeptides. One example of compounds that increase IMX129840 cytokine polypeptide activity are agonistic antibodies, preferably monoclonal antibodies, that bind to IMX129840 cytokine polypeptides or binding partners, which may increase IMX129840 cytokine polypeptide activity by causing constitutive intracellular signaling (or “ligand mimicking”), or by preventing the binding of a native inhibitor of IMX129840 cytokine polypeptide activity.

Antibodies to IMX129840 Cytokine Polypeptides

Antibodies that are immunoreactive with the polypeptides of the invention are provided herein. Such antibodies specifically bind to the polypeptides via the antigen-binding sites of the antibody (as opposed to non-specific binding). In the present invention, specifically binding antibodies are those that will specifically recognize and bind with IMX129840 cytokine polypeptides, homologues, and variants, but not with other molecules. In one preferred embodiment, the antibodies are specific for the polypeptides of the present invention and do not cross-react with other polypeptides. In this manner, the IMX129840 cytokine polypeptides, fragments, variants, fusion polypeptides, etc., as set forth above can be employed as “immunogens” in producing antibodies immunoreactive therewith.

More specifically, the polypeptides, fragment, variants, fusion polypeptides, etc. contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding (Janeway and Travers, Immuno Biology 3:9 (Garland Publishing Inc., 2nd ed. 1996)). Because folded polypeptides have complex surfaces, the number of epitopes available is quite numerous; however, due to the conformation of the polypeptide and steric hindrances, the number of antibodies that actually bind to the epitopes is less than the number of available epitopes (Janeway and Travers, Immuno Biology 2:14 (Garland Publishing Inc., 2nd ed. 1996)). Epitopes can be identified by any of the methods known in the art. Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kozbor et al., 1984, J. Immunol. 133:3001-3005; Cole et al., 1983, Proc. Natl. Acad. Sci. USA 80:2026-2030); and the EBV-hybridoma technique (Cole et al., 1985, Monoclonal Antibodies And Cancer Therapy, Alan R. Liss, Inc., pp. 77-96). Hybridoma cell lines that produce monoclonal antibodies specific for the polypeptides of the invention are also contemplated herein. Such hybridomas can be produced and identified by conventional techniques. The hybridoma producing the mAb of this invention can be cultivated in vitro or in vivo. Production of high titers of mAbs in vivo makes this the presently preferred method of production. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. Other techniques known to those of skill in the art, such as phage display or ribosome display methods, can be used to produce antibodies specific for particular IMX129840 cytokine polypeptide epitopes.

For the production of antibodies, various host animals can be immunized by injection with one or more of the following: a IMX129840 cytokine polypeptide, a fragment of a IMX129840 cytokine polypeptide, a functional equivalent of a IMX129840 cytokine polypeptide, or a mutant form of a IMX129840 cytokine polypeptide. Such host animals can include but are not limited to rabbits, guinea pigs, mice, and rats. Various adjuvants can be used to increase the immunologic response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies can be of any immunoglobulin class including IgG, IgM, IgE, IgA, IgD and any subclass thereof.

In addition, techniques developed for the production of “chimeric antibodies” (Takeda et al., 1985, Nature, 314: 452-454; Morrison et al., 1984, Proc Natl Acad Sci USA 81: 6851-6855; Boulianne et al., 1984, Nature 312: 643-646; Neuberger et al., 1985, Nature 314: 268-270) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region. The monoclonal antibodies of the present invention also include humanized versions of murine monoclonal antibodies. Such humanized antibodies can be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Useful techniques for humanizing antibodies are also discussed in U.S. Pat. No. 6,054,297. Procedures to generate antibodies transgenically can be found in GB 2,272,440, U.S. Pat. Nos. 5,569,825 and 5,545,806, and related patents. Preferably, for use in humans, the antibodies are human or humanized; techniques for creating such human or humanized antibodies are also well known and are commercially available from, for example, Medarex Inc. (Princeton, N.J.) and Abgenix Inc. (Fremont, Calif.). In another preferred embodiment, fully human antibodies for use in humans are produced by screening a phage display library of human antibody variable domains (Vaughan et al., 1998, Nat. Biotechnol. 16(6): 535-539; and U.S. Pat. No. 5,969,108).

Antigen-binding antibody fragments that recognize specific epitopes can be generated by known techniques. For example, such fragments include but are not limited to: the F(ab′)2 fragments which can be produced by pepsin digestion of the antibody molecule and the Fab fragments which can be generated by reducing the disulfide bridges of the (ab′)₂ fragments. Alternatively, Fab expression libraries can be constructed (Huse et al., 1989, Science, 246:1275-1281) to allow rapid and easy identification of monoclonal Fab fragments with the desired specificity. Techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can also be adapted to produce single chain antibodies against IMX129840 cytokine gene products. Single chain antibodies are formed by linking the heavy and light chain fragments of the Fv region via an amino acid bridge, resulting in a single chain polypeptide. Such single chain antibodies can also be useful intracellularly (i.e., as 'intrabodies), for example as described by Marasco et al. (J. Immunol. Methods 231:223-238, 1999) for genetic therapy in HIV infection. In addition, antibodies to the IMX129840 cytokine polypeptide can, in turn, be utilized to generate anti-idiotype antibodies that “mimic” the IMX129840 cytokine polypeptide and that may bind to the IMX129840 cytokine polypeptide's binding partners using techniques well known to those skilled in the art. (See, e.g., Greenspan & Bona, 1993, FASEB J 7(5):437-444; and Nissinoff, 1991, J. Immunol. 147(8):2429-2438).

Antibodies that are immunoreactive with the polypeptides of the invention include bispecific antibodies (i.e., antibodies that are immunoreactive with the polypeptides of the invention via a first antigen binding domain, and also immunoreactive with a different polypeptide via a second antigen binding domain). A variety of bispecific antibodies have been prepared, and found useful both in vitro and in vivo (see, for example, U.S. Pat. No. 5,807,706; and Cao and Suresh, 1998, Bioconjugate Chem 9: 635-644). Numerous methods of preparing bispecific antibodies are known in the art, including the use of hybrid-hybridomas such as quadromas, which are formed by fusing two differed hybridomas, and triomas, which are formed by fusing a hybridoma with a lymphocyte (Milstein and Cuello, 1983, Nature 305: 537-540; U.S. Pat. No. 4,474,893; and U.S. Pat. No. 6,106,833). U.S. Pat. No. 6,060,285 discloses a process for the production of bispecific antibodies in which at least the genes for the light chain and the variable portion of the heavy chain of an antibody having a first specificity are transfected into a hybridoma cell secreting an antibody having a second specificity. Chemical coupling of antibody fragments has also been used to prepare antigen-binding molecules having specificity for two different antigens (Brennan et al., 1985, Science 229: 81-83; Glennie et al., J. Immunol., 1987, 139:2367-2375; and U.S. Pat. No. 6,010,902). Bispecific antibodies can also be produced via recombinant means, for example, by using. the leucine zipper moieties from the Fos and Jun proteins (which preferentially form heterodimers) as described by Kostelny et al. (J. Immunol. 148:1547-4553; 1992). U.S. Pat. No. 5,582,996 discloses the use of complementary interactive domains (such as leucine zipper moieties or other lock and key interactive domain structures) to facilitate heterodimer formation in the production of bispecific antibodies. Tetravalent, bispecific molecules can be prepared by fusion of DNA encoding the heavy chain of an F(ab′)2 fragment of an antibody with either DNA encoding the heavy chain of a second F(ab′)2 molecule (in which the CH1 domain is replaced by a CH3 domain), or with DNA encoding a single chain FV fragment of an antibody, as described in U.S. Pat. No. 5,959,083. Expression of the resultant fusion genes in mammalian cells, together with the genes for the corresponding light chains, yields tetravalent bispecific molecules having specificity for selected antigens. Bispecific antibodies can also be produced as described in U.S. Pat. No. 5,807,706. Generally, the method involves introducing a protuberance (constructed by replacing small amino acid side chains with larger side chains) at the interface of a first polypeptide and a corresponding cavity (prepared by replacing large amino acid side chains with smaller ones) in the interface of a second polypeptide. Moreover, single-chain variable fragments (sFvs) have been prepared by covalently joining two variable domains; the resulting antibody fragments can form dimers or trimers, depending on the length of a flexible linker between the two variable domains (Kortt et al., 1997, Protein Engineering 10:423-433).

Screening procedures by which such antibodies can be identified are well known, and can involve immunoaffinity chromatography, for example. Antibodies can be screened for agonistic (i.e., ligand-mimicking) properties. Such antibodies, upon binding to cell surface IMX129840 cytokine, induce biological effects (e.g., transduction of biological signals) similar to the biological effects induced when the IMX129840 cytokine binding partner binds to cell surface IMX129840 cytokine. Agonistic antibodies can be used to induce IMX129840 cytokine-mediated cell stimulatory pathways or intercellular communication. Bispecific antibodies can be identified by screening with two separate assays, or with an assay wherein the bispecific antibody serves as a bridge between the first antigen and the second antigen (the latter is coupled to a detectable moiety). Bispecific antibodies that bind IMX129840 cytokine polypeptides of the invention via a first antigen binding domain will be useful in diagnostic applications and in treating conditions and diseases involving the proliferation or the development of cells from pluripotent stem cell precursors.

Those antibodies that can block binding of the IMX129840 cytokine polypeptides of the invention to binding partners for IMX129840 cytokine can be used to inhibit IMX129840 cytokine-mediated intercellular communication or cell stimulation that results from such binding. Such blocking antibodies can be identified using any suitable assay procedure, such as by testing antibodies for the ability to inhibit binding of IMX129840 cytokine to certain cells expressing an IMX129840 cytokine binding partner. Alternatively, blocking antibodies can be identified in assays for the ability to inhibit a biological effect that results from binding of soluble IMX129840 cytokine to target cells. Antibodies can be assayed for the ability to inhibit IMX129840 cytokine binding partner-mediated cell stimulatory pathways, for example. Such an antibody can be employed in an in vitro procedure, or administered in vivo to inhibit a biological activity mediated by the entity that generated the antibody. Disorders caused or exacerbated (directly or indirectly) by the interaction of IMX129840 cytokine with cell surface binding partner receptor thus can be treated. A therapeutic method involves in vivo administration of a blocking antibody to a mammal in an amount effective in inhibiting IMX129840 cytokine binding partner-mediated biological activity. Monoclonal antibodies are generally preferred for use in such therapeutic methods. In one embodiment, an antigen-binding antibody fragment is employed. Compositions comprising an antibody that is directed against IMX129840 cytokine, and a physiologically acceptable diluent, excipient, or carrier, are provided herein. Suitable components of such compositions are as described below for compositions containing IMX129840 cytokine polypeptides.

Also provided herein are conjugates comprising a detectable (e.g., diagnostic) or therapeutic agent, attached to the antibody. Examples of such agents are presented above. The conjugates find use in in vitro or in vivo procedures. The antibodies of the invention can also be used in assays to detect the presence of the polypeptides or fragments of the invention, either in vitro or in vivo. The antibodies also can be employed in purifying polypeptides or fragments of the invention by immunoaffinity chromatography.

Rational Design of Compounds that Interact with IMX129840 Cytokine Polypeptides

The goal of rational drug design is to produce structural analogs of biologically active polypeptides of interest or of small molecules with which they interact, e.g., inhibitors, agonists, antagonists, etc. Any of these examples can be used to fashion drugs which are more active or stable forms of the polypeptide or which enhance or interfere with the function of a polypeptide in vivo (Hodgson J (1991) Biotechnology 9:19-21). In one approach, the three-dimensional structure of a polypeptide of interest, or of a polypeptide-inhibitor complex, is determined by x-ray crystallography, by nuclear magnetic resonance, or by computer homology modeling or, most typically, by a combination of these approaches. Both the shape and charges of the polypeptide must be ascertained to elucidate the structure and to determine active site(s) of the molecule. Less often, useful information regarding the structure of a polypeptide may be gained by modeling based on the structure of homologous polypeptides. In both cases, relevant structural information is used to design analogous IMX129840 cytokine-like molecules, to identify efficient inhibitors, or to identify small molecules that bind IMX129840 cytokine polypeptides. Useful examples of rational drug design include molecules which have improved activity or stability as shown by Braxton S and Wells JA (1992 Biochemistry 31:7796-7801) or which act as inhibitors, agonists, or antagonists of native peptides as shown by Athauda SB et al (1993 J Biochem 113:742-746). The use of IMX129840 cytokine polypeptide structural information in molecular modeling software systems to assist in inhibitor design and in studying inhibitor-IMX129840 cytokine polypeptide interaction is also encompassed by the invention. A particular method of the invention comprises analyzing the three dimensional structure of IMX129840 cytokine polypeptides for likely binding sites of substrates, synthesizing a new molecule that incorporates a predictive reactive site, and assaying the new molecule as described further herein.

It is also possible to isolate a target-specific antibody, selected by functional assay, as described further herein, and then to solve its crystal structure. This approach, in principle, yields a pharmacore upon which subsequent drug design can be based. It is possible to bypass polypeptide crystallography altogether by generating anti-idiotypic antibodies (anti-ids) to a functional, pharmacologically active antibody. As a mirror image of a mirror image, the binding site of the anti-ids would be expected to be an analog of the original antigen. The anti-id could then be used to identify and isolate peptides from banks of chemically or biologically produced peptides. The isolated peptides would then act as the pharmacore.

Assays of IMX129840 Cytokine Polypeptide Activities

The purified IMX129840 cytokine polypeptides of the invention (including polypeptides, polypeptides, fragments, variants, oligomers, and other forms) are useful in a variety of assays. For example, the IMX129840 cytokine molecules of the present invention can be used to identify binding partners of IMX129840 cytokine polypeptides, which can also be used to modulate intercellular communication, cell stimulation, or immune cell activity. Alternatively, they can be used to identify non-binding-partner molecules or substances that modulate intercellular communication, cell stimulatory pathways, or immune cell activity.

Assays to Identify Binding Partners. Polypeptides of the IMX129840 cytokine family and fragments thereof can be used to identify binding partners. For example, they can be tested for the ability to bind a candidate binding partner in any suitable assay, such as a conventional binding assay. To illustrate, the IMX129840 cytokine polypeptide can be labeled with a detectable reagent (e.g., a radionuclide, chromophore, enzyme that catalyzes a colorimetric or fluorometric reaction, and the like). The labeled polypeptide is contacted with cells expressing the candidate binding partner. The cells then are washed to remove unbound labeled polypeptide, and the presence of cell-bound label is determined by a suitable technique, chosen according to the nature of the label.

One example of a binding assay procedure is as follows. A recombinant expression vector containing the candidate binding partner cDNA is constructed. CV1-EBNA-1 cells in 10 cm² dishes are transfected with this recombinant expression vector. CV-1/EBNA-1 cells (ATCC CRL 10478) constitutively express EBV nuclear antigen-1 driven from the CMV Immediate-early enhancer/promoter. CV1-EBNA-1 was derived from the African Green Monkey kidney cell line CV-1 (ATCC CCL 70), as described by McMahan et al., (EMBO J. 10:2821, 1991). The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated for 1 hour at 37° C. with various concentrations of, for example, a soluble polypeptide/Fc fusion polypeptide made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization. The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc polypeptide that has bound to the cells. In all assays, non-specific binding of ¹²⁵I-antibody is assayed in the absence of the Fc fusion polypeptide/Fc, as well as in the presence of the Fc fusion polypeptide and a 200-fold molar excess of unlabeled mouse anti-human IgG antibody. Cell-bound ¹²⁵I-antibody is quantified on a Packard Autogamma counter. Affinity calculations (Scatchard, Ann. N.Y. Acad. Sci. 51:660, 1949) are generated on RS/1 (BBN Software, Boston, Mass.) run on a Microvax computer. Binding can also be detected using methods that are well suited for high-throughput screening procedures, such as scintillation proximity assays (Udenfriend et al., 1985, Proc Natl Acad Sci USA 82: 8672-8676), homogeneous time-resolved fluorescence methods (Park et al., 1999, Anal Biochem 269: 94-104), fluorescence resonance energy transfer (FRET) methods (Clegg R M, 1995, Curr Opin Biotechnol 6: 103-110), or methods that measure any changes in surface plasmon resonance when a bound polypeptide is exposed to a potential binding partner, using for example a biosensor such as that supplied by Biacore AB (Uppsala, Sweden). Compounds that can be assayed for binding to IMX129840 cytokine polypeptides include but are not limited to small organic molecules, such as those that are commercially available—often as part of large combinatorial chemistry compound ‘libraries’—from companies such as Sigma-Aldrich (St. Louis, Mo.), Arqule (Woburn, Mass.), Enzymed (Iowa City, Iowa), Maybridge Chemical Co. (Trevillett, Cornwall, UK), MDS Panlabs (Bothell, Wash.), Pharmacopeia (Princeton, N.J.), and Trega (San Diego, Calif.). Preferred small organic molecules for screening using these assays are usually less than 10 K molecular weight and can possess a number of physicochemical and pharmacological properties which enhance cell penetration, resist degradation, and/or prolong their physiological half-lives (Gibbs, J., 1994, Pharmaceutical Research in Molecular Oncology, Cell 79(2): 193-198). Compounds including natural products, inorganic chemicals, and biologically active materials such as proteins and toxins can also be assayed using these methods for the ability to bind to IMX129840 cytokine polypeptides.

Yeast Two-Hybrid or “Interaction Trap” Assays. Where the IMX129840 cytokine polypeptide binds or potentially binds to another polypeptide (such as, for example, in a receptor-ligand interaction), the nucleic acid encoding the IMX129840 cytokine polypeptide can also be used in interaction trap assays (such as, for example, that described in Gyuris et al., Cell 75:791-803 (1993)) to identify nucleic acids encoding the other polypeptide with which binding occurs or to identify inhibitors of the binding interaction. Polypeptides involved in these binding interactions can also be used to screen for peptide or small molecule inhibitors or agonists of the binding interaction.

Competitive Binding Assays. Another type of suitable binding assay is a competitive binding assay. To illustrate, biological activity of a variant can be determined by assaying for the variant's ability to compete with the native polypeptide for binding to the candidate binding partner. Competitive binding assays can be performed by conventional methodology. Reagents that can be employed in competitive binding assays include radiolabeled IMX129840 cytokine and intact cells expressing IMX129840 cytokine (endogenous or recombinant) on the cell surface. For example, a radiolabeled soluble IMX129840 cytokine fragment can be used to compete with a soluble IMX129840 cytokine variant for binding to cell surface receptors. Instead of intact cells, one could substitute a soluble binding partner/Fc fusion polypeptide bound to a solid phase through the interaction of Polypeptide A or Polypeptide G (on the solid phase) with the Fc moiety. Chromatography columns that contain Polypeptide A and Polypeptide G include those available from Pharmacia Biotech, Inc., Piscataway, N.J.

Assays to Identify Modulators of Intercellular Communication. Cell Stimulation, or Immune Cell Activity. The influence of IMX129840 cytokine on intercellular communication, cell stimulation, or immune cell activity can be manipulated to control these activities in target cells. For example, the disclosed IMX129840 cytokine polypeptides, nucleic acids encoding the disclosed IMX129840 cytokine polypeptides, or agonists or antagonists of such polypeptides can be administered to a cell or group of cells to induce, enhance, suppress, or arrest cellular communication, cell stimulation, or activity in the target cells. Identification of IMX129840 cytokine polypeptides, agonists or antagonists that can be used in this manner can be carried out via a variety of assays known to those skilled in the art. Included in such assays are those that evaluate the ability of an IMX129840 cytokine polypeptide to influence intercellular communication, cell stimulation or activity. Such an assay would involve, for example, the analysis of immune cell interaction in the presence of an IMX129840 cytokine polypeptide. In such an assay, one would determine a rate of communication or cell stimulation in the presence of the IMX129840 cytokine polypeptide and then determine if such communication or cell stimulation is altered in the presence of a candidate agonist or antagonist or another IMX129840 cytokine polypeptide. Exemplary assays for this aspect of the invention include cytokine secretion assays, T-cell co-stimulation assays, and mixed lymphocyte reactions involving antigen presenting cells and T cells. These assays are well known to those skilled in the art.

In another aspect, the present invention provides a method of detecting the ability of a test compound to affect the intercellular communication or cell stimulatory activity of a cell. In this aspect, the method comprises: (1) contacting a first group of target cells with a test compound including an IMX129840 cytokine receptor polypeptide or fragment thereof under conditions appropriate to the particular assay being used; (2) measuring the net rate of intercellular communication or cell stimulation among the target cells; and (3) observing the net rate of intercellular communication or cell stimulation among control cells contacted with the IMX129840 cytokine receptor polypeptides or fragments thereof, in the absence of a test compound, under otherwise identical conditions as the first group of cells. In this embodiment, the net rate of intercellular communication or cell stimulation in the control cells is compared to that of the cells treated with both the IMX129840 cytokine molecule as well as a test compound. The comparison will provide a difference in the net rate of intercellular communication or cell stimulation such that an effector of intercellular communication or cell stimulation can be identified. The test compound can function as an effector by either activating or up-regulating, or by inhibiting or down-regulating intercellular communication or cell stimulation, and can be detected through this method.

Cell Proliferation, Cell Death, Cell Differentiation, and Cell Adhesion Assays. A polypeptide of the present invention may exhibit cytokine, cell proliferation (either inducing or inhibiting), or cell differentiation (either inducing or inhibiting) activity, or may induce production of other cytokines in certain cell populations. Many polypeptide factors discovered to date have exhibited such activity in one or more factor-dependent cell proliferation assays, and hence the assays serve as a convenient confirmation of cell stimulatory activity. The activity of a polypeptide of the present invention is evidenced by any one of a number of routine factor-dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK. The activity of a IMX129840 cytokine polypeptide of the invention may, among other means, be measured by the following methods:

Assays for T-cell or thymocyte proliferation include without limitation those described in: Current Protocols in Immunology, Coligan et al. eds, Greene Publishing Associates and Wiley-Interscience (pp. 3.1-3.19: In vitro assays for mouse lymphocyte function; Chapter 7: Immunologic studies in humans); Takai et al., J. Immunol. 137: 3494-3500, 1986; Bertagnolli et al., J. Immunol. 145: 1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Kruisbeek and Shevach, 1994, Polyclonal T cell stimulation, in Current Protocols in Immunology, Coligan et al. eds. Vol 1 pp. 3.12.1-3.12.14, John Wiley and Sons, Toronto; and Schreiber, 1994, Measurement of mouse and human interferon gamma in Current Protocols in Immunology, Coligan et al. eds. Vol 1 pp. 6.8.1-6.8.8, John Wiley and Sons, Toronto.

Assays for proliferation and differentiation of hematopoietic and lymphopoietic cells include, without limitation, those described in: Bottomly et al., 1991, Measurement of human and murine interleukin 2 and interleukin 4, in Current Protocols in Immunology, Coligan et al. eds. Vol 1 pp. 6.3.1-6.3.12, John Wiley and Sons, Toronto; deVries et al., J Exp Med 173: 1205-1211, 1991; Moreau et al., Nature 336:690-692, 1988; Greenberger et al., Proc Natl Acad. Sci. USA 80: 2931-2938, 1983; Nordan, 1991, Measurement of mouse and human interleukin 6, in Current Protocols in Immunology Coligan et al. eds. Vol 1 pp. 6.6.1-6.6.5, John Wiley and Sons, Toronto; Smith et al., Proc Natl Acad Sci USA 83: 1857-1861, 1986; Bennett et al., 1991, Measurement of human interleukin 11, in Current Protocols in Immunology Coligan et al. eds. Vol 1 pp. 6.15.1 John Wiley and Sons, Toronto; Ciarletta et al., 1991, Measurement of mouse and human Interleukin 9, in Current Protocols in Immunology Coligan et al. eds. Vol 1 pp. 6.13.1, John Wiley and Sons, Toronto.

Assays for T-cell clone responses to antigens (which will identify, among others, polypeptides that affect APC-T cell interactions as well as direct T-cell effects by measuring proliferation and cytokine production) include, without limitation, those described in: Current Protocols in Immunology, Coligan et al. eds, Greene Publishing Associates and Wiley-Interscience (Chapter 3: In vitro assays for mouse lymphocyte function; Chapter 6: Cytokines and their cellular receptors; Chapter 7: Immunologic studies in humans); Weinberger et al., Proc Natl Acad Sci USA 77: 6091-6095, 1980; Weinberger et al., Eur. J. Immun. 11:405-411, 1981; Takai et al., J. Immunol. 137:3494-3500, 1986; Takai et al., J. Immunol. 140:508-512, 1988

Assays for thymocyte or splenocyte cytotoxicity include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, polypeptides that modulate T-cell dependent antibody responses and that affect Th1/Th2 profiles) include, without limitation, those described in: Maliszewski, J Immunol 144: 3028-3033, 1990; and Mond and Brunswick, 1994, Assays for B cell function: in vitro antibody production, in Current Protocols in Immunology Coligan et al. eds. Vol 1 pp. 3.8.1-3.8.16, John Wiley and Sons, Toronto.

Mixed lymphocyte reaction (MLR) assays (which will identify, among others, polypeptides that generate predominantly Th1 and CTL responses) include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, polypeptides expressed by dendritic cells that activate naive T-cells) include, without limitation, those described in: Guery et al., J. Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virology 67:4062-4069, 1993; Huang et al., Science 264:961-965, 1994; Macatonia et al., J Exp Med 169:1255-1264, 1989; Bhardwaj et al., J Clin Invest 94:797-807, 1994; and Inaba et al., J Exp Med 172:631-640,1990.

Assays for lymphocyte survival/apoptosis (which will identify, among others, polypeptides that prevent apoptosis after superantigen induction and polypeptides that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., International Journal of Oncology 1:639-648, 1992.

Assays for polypeptides that influence early steps of T-cell commitment and development include, without limitation, those described in: Antica et al., Blood 84:111-117, 1994; Fine et al., Cell Immunol 155:111-122, 1994; Galy et al., Blood 85:2770-2778, 1995; Toki et al., Proc Natl Acad. Sci. USA 88:7548-7551, 1991

Assays for embryonic stem cell differentiation (which will identify, among others, polypeptides that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, polypeptides that regulate lympho-hematopoiesis) include, without limitation, those described in: Methylcellulose colony forming assays, Freshney, 1994, In Culture of Hematopoietic Cells, Freshney et al. eds. pp. 265-268, Wiley-Liss, Inc., New York, N.Y.; Hirayama et al., Proc. Natl. Acad. Sci. USA 89:5907-5911, 1992; Primitive hematopoietic colony forming cells with high proliferative potential, McNiece and Briddell, 1994, In Culture of Hematopoietic Cells, Freshney et al. eds. pp. 23-39, Wiley-Liss, Inc., New York, N.Y.; Neben et al., Experimental Hematology 22:353-359, 1994; Ploemacher, 1994, Cobblestone area forming cell assay, In Culture of Hematopoietic Cells, Freshney et al. eds. pp. 1-21, Wiley-Liss, Inc., New York, N.Y.; Spooncer et al., 1994, Long term bone marrow cultures in the presence of stromal cells, In Culture of Hematopoietic Cells, Freshney et al. eds. pp. 163-179, Wiley-Liss, Inc., New York, N.Y.; Sutherland, 1994, Long term culture initiating cell assay, In Culture of Hematopoietic Cells, Freshney et al. eds. Vol pp. 139-162, Wiley-Liss, Inc., New York, N.Y.

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium). Assays for wound healing activity include, without limitation, those described in: Winter, Epidermal Wound Healing, pps. 71-112 (Maibach and Rovee, eds.), Year Book Medical Publishers, Inc., Chicago, as modified by Eaglstein and Mertz, J. Invest. Dermatol 71:382-84 (1978).

Assays for cell movement and adhesion include, without limitation, those described in: Current Protocols in Immunology Coligan et al. eds, Greene Publishing Associates and Wiley-Interscience (Chapter 6.12, Measurement of alpha and beta cytokines 6.12.1-6.12.28); Taub et al. J. Clin. Invest. 95:1370-1376, 1995; Lind et al. APMIS 103:140-146, 1995; Muller et al Eur. J. Immunol. 25: 1744-1748; Gruber et al. J. Immunol. 152:5860-5867, 1994; Johnston et al. J. Immunol. 153: 1762-1768, 1994

Assays for receptor-ligand activity include without limitation those described in: Current Protocols in Immunology Coligan et al. eds, Greene Publishing Associates and Wiley-Interscience (Chapter 7.28, Measurement of cellular adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Diagnostic and Other Uses of IMX129840 Cytokine Polypeptides and Nucleic Acids

The nucleic acids encoding the IMX129840 cytokine polypeptides provided by the present invention can be used for numerous diagnostic or other useful purposes. The nucleic acids of the invention can be used to express recombinant polypeptide for analysis, characterization or therapeutic use; as markers for tissues in which the corresponding polypeptide is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel nucleic acids; for selecting and making oligomers for attachment to a “gene chip” or other support, including for examination of expression patterns; to raise anti-polypeptide antibodies using DNA immunization techniques; as an antigen to raise anti-DNA antibodies or elicit another immune response, and. for gene therapy. Uses of IMX129840 cytokine polypeptides and fragmented polypeptides include, but are not limited to, the following: purifying polypeptides and measuring the activity thereof; delivery agents; therapeutic and research reagents; molecular weight and isoelectric focusing markers; controls for peptide fragmentation; identification of unknown polypeptides; and preparation of antibodies. Any or all nucleic acids suitable for these uses are capable of being developed into reagent grade or kit format for commercialization as products. Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987

Probes and Primers. Among the uses of the disclosed IMX129840 cytokine nucleic acids, and combinations of fragments thereof, is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence. The basic parameters affecting the choice of hybridization conditions and guidance for devising suitable conditions are set forth by Sambrook et al., 1989 and are described in detail above. Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified. In certain embodiments, degenerate primers can be used as probes for non-human genetic libraries. Such libraries would include but are not limited to cDNA libraries, genomic libraries, and even electronic EST (express sequence tag) or DNA libraries. Homologous sequences identified by this method would then be used as probes to identify non-human IMX129840 cytokine homologues.

Chromosome Mapping. The nucleic acids encoding IMX129840 cytokine polypeptides, and the disclosed fragments and combinations of these nucleic acids, can be used by those skilled in the art using well-known techniques to identify the human chromosome to which these nucleic acids map. Useful techniques include, but are not limited to, using the sequence or portions, including oligonucleotides, as a probe in various well-known techniques such as radiation hybrid mapping (high resolution), in situ hybridization to chromosome spreads (moderate resolution), and Southern blot hybridization to hybrid cell lines containing individual human chromosomes (low resolution). For example, chromosomes can be mapped by radiation hybridization. PCR is performed using the Whitehead Institute/MIT Center for Genome Research Genebridge4 panel of 93 radiation hybrids, using primers that lie within a putative exon of the gene of interest and which amplify a product from human genomic DNA, but do not amplify hamster genomic DNA. The PCR results are converted into a data vector that is submitted to the Whitehead/MIT Radiation Mapping site (www-seq.wi.mit.edu). The data is scored and the chromosomal assignment and placement relative to known Sequence Tag Site (STS) markers on the radiation hybrid map is provided. Alternatively, the genomic sequences corresponding to nucleic acids encoding a IMX129840 cytokine polypeptide are mapped by comparison to sequences in public and proprietary databases, such as the GenBank non-redundant database (ncbi.nlm.nih.gov/BLAST), Locuslink (ncbi.nlm.nih.gov:80/LocusLink/), Unigene (ncbi.nlm.nih.gov/cgi-bin/UniGene), AceView (ncbi.nlm.nih.gov/AceView), Online Mendelian Inheritance in Man (OMIM) (ncbi.nlm.nih.gov/Omim), Gene Map Viewer (ncbi.nlm.nih.gov/genemap), and proprietary databases such as the Celera Discovery System (celera.com). These computer analyses of available genomic sequence information can provide the identification of the specific chromosomal location of human genomic sequences corresponding to sequences encoding human IMX129840 cytokine polypeptides, and the unique genetic mapping relationships between the IMX129840 cytokine genomic sequences and the genetic map locations of known human genetic disorders.

Diagnostics and Gene Therapy. The nucleic acids encoding IMX129840 cytokine polypeptides, and the disclosed fragments and combinations of these nucleic acids can be used by one skilled in the art using well-known techniques to analyze abnormalities associated with the genes corresponding to these polypeptides. This enables one to distinguish conditions in which this marker is rearranged or deleted. In addition, nucleic acids of the invention or a fragment thereof can be used as a positional marker to map other genes of unknown location. The DNA can be used in developing treatments for any disorder mediated (directly or indirectly) by defective, or insufficient amounts of, the genes corresponding to the nucleic acids of the invention. Disclosure herein of native nucleotide sequences permits the detection of defective genes, and the replacement thereof with normal genes. Defective genes can be detected in in vitro diagnostic assays, and by comparison of a native nucleotide sequence disclosed herein with that of a gene derived from a person suspected of harboring a defect in this gene.

Methods of Screening for Binding Partners. The IMX129840 cytokine polypeptides of the invention each can be used as reagents in methods to screen for or identify binding partners. For example, the IMX129840 cytokine polypeptides can be attached to a solid support material and may bind to their binding partners in a manner similar to affinity chromatography. In particular embodiments, a polypeptide is attached to a solid support by conventional procedures. As one example, chromatography columns containing functional groups that will react with functional groups on amino acid side chains of polypeptides are available (Pharmacia Biotech, Inc., Piscataway, N.J.). In an alternative, a polypeptide/Fc polypeptide (as discussed above) is attached to protein A- or protein G-containing chromatography columns through interaction with the Fc moiety. The IMX129840 cytokine polypeptides also find use in identifying cells that express a IMX129840 cytokine binding partner on the cell surface. Purified IMX129840 cytokine polypeptides are bound to a solid phase such as a column chromatography matrix or a similar suitable substrate. For example, magnetic microspheres can be coated with the polypeptides and held in an incubation vessel through a magnetic field. Suspensions of cell mixtures containing potential binding-partner-expressing cells are contacted with the solid phase having the polypeptides thereon. Cells expressing the binding partner on the cell surface bind to the fixed polypeptides, and unbound cells are washed away. Alternatively, IMX129840 cytokine polypeptides can be conjugated to a detectable moiety, then incubated with cells to be tested for binding partner expression. After incubation, unbound labeled matter is removed and the presence or absence of the detectable moiety on the cells is determined. In a further alternative, mixtures of cells suspected of expressing the binding partner are incubated with biotinylated polypeptides. Incubation periods are typically at least one hour in duration to ensure sufficient binding. The resulting mixture then is passed through a column packed with avidin-coated beads, whereby the high affinity of biotin for avidin provides binding of the desired cells to the beads. Procedures for using avidin-coated beads are known (see Berenson, et al. J. Cell. Biochem., 10D:239, 1986). Washing to remove unbound material, and the release of the bound cells, are performed using conventional methods. In some instances, the above methods for screening for or identifying binding partners may also be used or modified to isolate or purify such binding partner molecules or cells expressing them.

Measuring Biological Activity. Polypeptides also find use in measuring the biological activity of IMX129840 cytokine-binding polypeptides in terms of their binding affinity. The polypeptides thus can be employed by those conducting “quality assurance” studies, e.g., to monitor shelf life and stability of polypeptide under different conditions. For example, the polypeptides can be employed in a binding affinity study to measure the biological activity of a binding partner polypeptide that has been stored at different temperatures, or produced in different cell types. The polypeptides also can be used to determine whether biological activity is retained after modification of a binding partner polypeptide (e.g., chemical modification, truncation, mutation, etc.). The binding affinity of the modified polypeptide is compared to that of an unmodified binding polypeptide to detect any adverse impact of the modifications on biological activity of the binding polypeptide. The biological activity of a binding polypeptide thus can be ascertained before it is used in a research study, for example.

Carriers and Delivery Agents. The polypeptides also find use as carriers for delivering agents attached thereto to cells bearing identified binding partners. The polypeptides thus can be used to deliver diagnostic or therapeutic agents to such cells (or to other cell types found to express binding partners on the cell surface) in in vitro or in vivo procedures. Detectable (diagnostic) and therapeutic agents that can be attached to a polypeptide include, but are not limited to, toxins, other cytotoxic agents, drugs, radionuclides, chromophores, enzymes that catalyze a colorimetric or fluorometric reaction, and the like, with the particular agent being chosen according to the intended application. Among the toxins are ricin, abrin, diphtheria toxin, Pseudomonas aeruginosa exotoxin A, ribosomal inactivating polypeptides, mycotoxins such as trichothecenes, and derivatives and fragments (e.g., single chains) thereof. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are ¹³¹I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu. Such agents can be attached to the polypeptide by any suitable conventional procedure. The polypeptide comprises functional groups on amino acid side chains that can be reacted with functional groups on a desired agent to form covalent bonds, for example. Alternatively, the polypeptide or agent can be derivatized to generate or attach a desired reactive functional group. The derivatization can involve attachment of one of the bifunctional coupling reagents available for attaching various molecules to polypeptides (Pierce Chemical Company, Rockford, Ill.). A number of techniques for radiolabeling polypeptides are known. Radionuclide metals can be attached to polypeptides by using a suitable bifunctional chelating agent. Conjugates comprising polypeptides and a suitable diagnostic or therapeutic agent (preferably covalently linked) are thus prepared. The conjugates are administered or otherwise employed in an amount appropriate for the particular application.

Treating Diseases with IMX129840 Cytokine Polypeptides and Antagonists Thereof

The IMX129840 cytokine polypeptides, fragments, variants, antagonists, agonists, antibodies, and binding partners of the invention are likely to be useful for treating medical conditions and diseases including, but not limited to, conditions and diseases involving the proliferation or the development of cells from pluripotent stem cell precursors. The therapeutic molecule or molecules to be used will depend on the etiology of the condition to be treated and the biological pathways involved, and variants, fragments, and binding partners of IMX129840 cytokine polypeptides may have effects similar to or different from IMX129840 cytokine polypeptides. For example, an antagonist of the stimulation of cell proliferation activity of IMX129840 cytokine polypeptides can be selected for treatment of conditions involving excess proliferation and/or differentiation of cells from pluripotent stem cell precursors, but a particular fragment of a given IMX129840 cytokine polypeptide may also act as an effective dominant negative antagonist of that activity. As another example, it is known that cytokines such as interferon-gamma increase epithelial barrier function in certain cell types such as lung epithelia, and decrease epithelial barrier function in other cell types such as intestinal epithelia. Therefore, in the following paragraphs “IMX129840 cytokine polypeptides or antagonists” refers to all IMX129840 cytokine polypeptides, fragments, variants, antagonists, agonists, antibodies, and binding partners etc. of the invention, and it is understood that a specific molecule or molecules can be selected from those provided as embodiments of the invention by individuals of skill in the art, according to the biological and therapeutic considerations described herein.

IMX129840 cytokine polypeptides or antagonists are useful in the treatment of disorders involving inflammation and/or excess cell proliferation. Certain conditions of the gastrointestinal system are treatable with IMX129840 cytokine polypeptides or antagonists or combination therapies, including Crohn's disease and ulcerative colitis. A number of pulmonary disorders also can be treated with the disclosed IMX129840 cytokine polypeptides or antagonists and combination therapies, such as allergies, including allergic rhinitis, contact dermatitis, atopic dermatitis, and asthma. Disorders involving the skin or mucous membranes also are treatable using the disclosed cytokine polypeptides or antagonists, compositions or combination therapies. Such disorders include inflammatory skin diseases and hyperproliferative disorders such as, for example, psoriasis.

Administration of IMX129840 Cytokine Polypeptides and Antagonists Thereof

This invention provides compounds, compositions, and methods for treating a patient, preferably a mammalian patient, and most preferably a human patient, who is suffering from a medical disorder, and in particular a IMX129840 cytokine-mediated disorder. Such IMX129840 cytokine-mediated disorders include conditions caused (directly or indirectly) or exacerbated by binding between IMX129840 cytokine and a binding partner. For purposes of this disclosure, the terms “illness,” “disease,” “medical condition,” “abnormal condition” and the like are used interchangeably with the term “medical disorder.” The terms “treat”, “treating”, and “treatment” used herein includes curative, preventative (e.g., prophylactic) and palliative or ameliorative treatment. For such therapeutic uses, IMX129840 cytokine polypeptides and fragments, IMX129840 cytokine nucleic acids encoding the IMX129840 cytokine family polypeptides, and/or agonists or antagonists of the IMX129840 cytokine polypeptide such as antibodies can be administered to the patient in need through well-known means. Compositions of the present invention can contain a polypeptide in any form described herein, such as native polypeptides, variants, derivatives, oligomers, and biologically active fragments. In particular embodiments, the composition comprises a soluble polypeptide or an oligomer comprising soluble IMX129840 cytokine polypeptides.

Therapeutically Effective Amount. In practicing the method of treatment or use of the present invention, a therapeutically effective amount of a therapeutic agent of the present invention is administered to a patient having a condition to be treated, preferably to treat or ameliorate diseases associated with the activity of a IMX129840 cytokine family polypeptide. “Therapeutic agent” includes without limitation any of the IMX129840 cytokine polypeptides, fragments, and variants; nucleic acids encoding the IMX129840 cytokine family polypeptides, fragments, and variants; agonists or antagonists of the IMX129840 cytokine polypeptides such as antibodies; IMX129840 cytokine polypeptide binding partners; complexes formed from the IMX129840 cytokine family polypeptides, fragments, variants, and binding partners, etc. As used herein, the term “therapeutically effective amount” means the total amount of each therapeutic agent or other active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual therapeutic agent or active ingredient, administered alone, the term refers to that ingredient alone. When applied to a combination, the term refers to combined amounts of the ingredients that result in the therapeutic effect, whether administered in combination, serially or simultaneously. As used herein, the phrase “administering a therapeutically effective amount” of a therapeutic agent means that the patient is treated with said therapeutic agent in an amount and for a time sufficient to induce an improvement, and preferably a sustained improvement, in at least one indicator that reflects the severity of the disorder. An improvement is considered “sustained” if the patient exhibits the improvement on at least two occasions separated by one or more days, or more preferably, by one or more weeks. The degree of improvement is determined based on signs or symptoms, and determinations can also employ questionnaires that are administered to the patient, such as quality-of-life questionnaires. Various indicators that reflect the extent of the patient's illness can be assessed for determining whether the amount and time of the treatment is sufficient. The baseline value for the chosen indicator or indicators is established by examination of the patient prior to administration of the first dose of the therapeutic agent. Preferably, the baseline examination is done within about 60 days of administering the first dose. If the therapeutic agent is being administered to treat acute symptoms, the first dose is administered as soon as practically possible after the injury has occurred. Improvement is induced by administering therapeutic agents such as IMX129840 cytokine polypeptides or antagonists until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating injuries or other acute conditions. Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Dosing. One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention can be administered until the optimal therapeutic effect is obtained for the patient, and at that point the dosage is not increased further. It is contemplated that the various pharmaceutical compositions used to practice the method of the present invention should contain about 0.01 ng to about 100 mg (preferably about 0.1 ng to about 10 mg, more preferably about 0.1 microgram to about 1 mg) of polypeptide of the present invention per kg body weight. In one embodiment of the invention, IMX129840 cytokine polypeptides or antagonists are administered one time per week to treat the various medical disorders disclosed herein, in another embodiment is administered at least two times per week, and in another embodiment is administered at least three times per week. If injected, the effective amount of IMX129840 cytokine polypeptides or antagonists per adult dose ranges from 1-20 mg/m², and preferably is about 5-12 mg/m². Alternatively, a flat dose can be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing IMX129840 cytokine polypeptides or antagonists at 25 mg/dose, or alternatively, containing 50 mg per dose. The 25 mg or 50 mg dose can be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of IMX129840 cytokine polypeptides or antagonists one to three times per week over a period of at least three weeks, or a dose of 50 mg of IMX129840 cytokine polypeptides or antagonists one or two times per week for at least three weeks, though treatment for longer periods may be necessary to induce the desired degree of improvement. For incurable chronic conditions, the regimen can be continued indefinitely, with adjustments being made to dose and frequency if such are deemed necessary by the patient's physician. The foregoing doses are examples for an adult patient who is a person who is 18 years of age or older. For pediatric patients (age 4-17), a suitable regimen involves the subcutaneous injection of 0.4 mg/kg, up to a maximum dose of 25 mg of IMX129840 cytokine polypeptides or antagonists, administered by subcutaneous injection one or more times per week. If an antibody against a IMX129840 cytokine polypeptide is used as the IMX129840 cytokine polypeptide antagonist, a preferred dose range is 0.1 to 20 mg/kg, and more preferably is 1-10 mg/kg. Another preferred dose range for an anti-IMX129840 cytokine polypeptide antibody is 0.75 to 7.5 mg/kg of body weight. Humanized antibodies are preferred, that is, antibodies in which only the antigen-binding portion of the antibody molecule is derived from a non-human source. Such antibodies can be injected or administered intravenously.

Formulations. Compositions comprising an effective amount of a IMX129840 cytokine polypeptide of the present invention (from whatever source derived, including without limitation from recombinant and non-recombinant sources), in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington 's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. Such compositions will influence the physical state, solubility, stability, rate of in vivo release, and rate of in vivo clearance, and are thus chosen according to the intended application, so that the characteristics of the carrier will depend on the selected route of administration. In one preferred embodiment of the invention, sustained-release forms of IMX129840 cytokine polypeptides are used. Sustained-release forms suitable for use in the disclosed methods include, but are not limited to, IMX129840 cytokine polypeptides that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

Combinations of Therapeutic Compounds. A IMX129840 cytokine polypeptide of the present invention may be active in multimers (e.g., heterodimers or homodimers) or complexes with itself or other polypeptides. As a result, pharmaceutical compositions of the invention may comprise a polypeptide of the invention in such multimeric or complexed form. The pharmaceutical composition of the invention may be in the form of a complex of the polypeptide(s) of present invention along with polypeptide or peptide antigens. The invention further includes the administration of IMX129840 cytokine polypeptides or antagonists concurrently with one or more other drugs that are administered to the same patient in combination with the IMX129840 cytokine polypeptides or antagonists, each drug being administered according to a regimen suitable for that medicament. “Concurrent administration” encompasses simultaneous or sequential treatment with the components of the combination, as well as regimens in which the drugs are alternated, or wherein one component is administered long-term and the other(s) are administered intermittently. Components can be administered in the same or in separate compositions, and by the same or different routes of administration. Examples of components that can be administered concurrently with the pharmaceutical compositions of the invention are: cytokines, lymphokines, or other hematopoietic factors such as M-CSF, GM-CSF, TNF, IL-1, IL-2, IL-3, IL4, IL-5, IL-6, IL-7, IL-8, IL-9, IL-10, IL-11, IL-12, IL-13, IL-14, IL-15, IL-17, IL-18, IFN, TNF0, TNF1, TNF2, G-CSF, Meg-CSF, thrombopoietin, stem cell factor, and erythropoietin, or inhibitors or antagonists of any of these factors. The pharmaceutical composition can further contain other agents which either enhance the activity of the polypeptide or compliment its activity or use in treatment. Such additional factors and/or agents may be included in the pharmaceutical composition to produce a synergistic effect with polypeptide of the invention, or to minimize side effects. Conversely, a IMX129840 cytokine polypeptide or antagonist of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. Additional examples of drugs to be administered concurrently include but are not limited to antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose. Additionally, IMX129840 cytokine polypeptides or antagonists can be combined with a second IMX129840 cytokine polypeptide/antagonist, including an antibody against a IMX129840 cytokine polypeptide, or a IMX129840 cytokine polypeptide-derived peptide that acts as a competitive inhibitor of a native IMX129840 cytokine polypeptide.

Routes of Administration. Any efficacious route of administration can be used to therapeutically administer IMX129840 cytokine polypeptides or antagonists thereof, including those compositions comprising nucleic acids. Parenteral administration includes injection, for example, via intra-articular, intravenous, intramuscular, intralesional, intraperitoneal or subcutaneous routes by bolus injection or by continuous infusion, and also includes localized administration, e.g., at a site of disease or injury. Other suitable means of administration include sustained release from implants; aerosol inhalation and/or insufflation; eyedrops; vaginal or rectal suppositories; buccal preparations; oral preparations, including pills, syrups, lozenges, ice creams, or chewing gum; and topical preparations such as lotions, gels, sprays, ointments or other suitable techniques. Alternatively, polypeptideaceous IMX129840 cytokine polypeptides or antagonists may be administered by implanting cultured cells that express the polypeptide, for example, by implanting cells that express IMX129840 cytokine polypeptides or antagonists. Cells may also be cultured ex vivo in the presence of polypeptides of the present invention in order to modulate cell proliferation or to produce a desired effect on or activity in such cells. Treated cells can then be introduced in vivo for therapeutic purposes. The polypeptide of the instant invention may also be administered by the method of protein transduction. In this method, the IMX129840 cytokine polypeptide is covalently linked to a protein-transduction domain (PTD) such as, but not limited to, TAT, Antp, or VP22 (Schwarze et al., 2000, Cell Biology 10: 290-295). The PTD-linked peptides can then be transduced into cells by adding the peptides to tissue-culture media containing the cells (Schwarze et al., 1999, Science 285: 1569; Lindgren et al., 2000, TiPS 21: 99; Derossi et al., 1998, Cell Biology 8: 84; WO 00/34308; WO 99/29721; and WO 99/10376). In another embodiment, the patient's own cells are induced to produce IMX129840 cytokine polypeptides or antagonists by transfection in vivo or ex vivo with a DNA that encodes IMX129840 cytokine polypeptides or antagonists. This DNA can be introduced into the patient's cells, for example, by injecting naked DNA or liposome-encapsulated DNA that encodes IMX129840 cytokine polypeptides or antagonists, or by other means of transfection. Nucleic acids of the invention can also be administered to patients by other known methods for introduction of nucleic acid into a cell or organism (including, without limitation, in the form of viral vectors or naked DNA). When IMX129840 cytokine polypeptides or antagonists are administered in combination with one or more other biologically active compounds, these can be administered by the same or by different routes, and can be administered simultaneously, separately or sequentially.

Oral Administration. When a therapeutically effective amount of polypeptide of the present invention is administered orally, polypeptide of the present invention will be in the form of a tablet, capsule, powder, solution or elixir. When administered in tablet form, the pharmaceutical composition of the invention can additionally contain a solid carrier such as a gelatin or an adjuvant. The tablet, capsule, and powder contain from about 5 to 95% polypeptide of the present invention, and preferably from about 25 to 90% polypeptide of the present invention. When administered in liquid form, a liquid carrier such as water, petroleum, oils of animal or plant origin such as peanut oil, mineral oil, soybean oil, or sesame oil, or synthetic oils can be added. The liquid form of the pharmaceutical composition can further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of polypeptide of the present invention, and preferably from about 1 to 50% polypeptide of the present invention.

Intravenous Administration. When a therapeutically effective amount of polypeptide of the present invention is administered by intravenous, cutaneous or subcutaneous injection, polypeptide of the present invention will be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention can also contain stabilizers, preservatives, buffers, antioxidants, or other additives known to those of skill in the art. The duration of intravenous therapy using the pharmaceutical composition of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical composition of the present invention.

Bone and Tissue Administration. For compositions of the present invention which are useful for bone, cartilage, tendon or ligament disorders, the therapeutic method includes administering the composition topically, systematically, or locally as an implant or device. When administered, the therapeutic composition for use in this invention is, of course, in a pyrogen-free, physiologically acceptable form. Further, the composition can desirably be encapsulated or injected in a viscous form for delivery to the site of bone, cartilage or tissue damage. Topical administration may be suitable for wound healing and tissue repair. Therapeutically useful agents other than a polypeptide of the invention which may also optionally be included in the composition as described above, can alternatively or additionally, be administered simultaneously or sequentially with the composition in the methods of the invention. Preferably for bone and/or cartilage formation, the composition would include a matrix capable of delivering the polypeptide-containing composition to the site of bone and/or cartilage damage, providing a structure for the developing bone and cartilage and optimally capable of being resorbed into the body. Such matrices can be formed of materials presently in use for other implanted medical applications. The choice of matrix material is based on biocompatibility, biodegradability, mechanical properties, cosmetic appearance and interface properties. The particular application of the compositions will define the appropriate formulation. Potential matrices for the compositions can be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure polypeptides or extracellular matrix components. Other potential matrices are nonbiodegradable and chemically defined, such as sintered hydroxapatite, bioglass, aluminates, or other ceramics Matrices can be comprised of combinations of any of the above mentioned types of material, such as polylactic acid and hydroxyapatite or collagen and tricalciumphosphate. The bioceramics can be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the polypeptide compositions from disassociating from the matrix. A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethyl-cellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the polypeptide from the polymer matrix and to provide appropriate handling of the composition, yet not so much that the progenitor cells are prevented from infiltrating the matrix, thereby providing the polypeptide the opportunity to assist the osteogenic activity of the progenitor cells. In further compositions, polypeptides of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-alpha and TGF-beta), and insulin-like growth factor (IGF). The therapeutic compositions are also presently valuable for veterinary applications. Particularly domestic animals and thoroughbred horses, in addition to humans, are desired patients for such treatment with polypeptides of the present invention. The dosage regimen of a polypeptide-containing pharmaceutical composition to be used in tissue regeneration will be determined by the attending physician considering various factors which modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage can vary with the type of matrix used in the reconstitution and with inclusion of other polypeptides in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Veterinary Uses. In addition to human patients, IMX129840 cytokine polypeptides and antagonists are useful in the treatment of disease conditions in non-human animals, such as pets (dogs, cats, birds, primates, etc.), domestic farm animals (horses cattle, sheep, pigs, birds, etc.), or any animal that suffers from a IMX129840 cytokine-mediated condition. In such instances, an appropriate dose can be determined according to the animal's body weight. For example, a dose of 0.2-1 mg/kg may be used. Alternatively, the dose is determined according to the animal's surface area, an exemplary dose ranging from 0.1-20 mg/m², or more preferably, from 5-12 mg/m². For small animals, such as dogs or cats, a suitable dose is 0.4 mg/kg. In a preferred embodiment, IMX129840 cytokine polypeptides or antagonists (preferably constructed from genes derived from the same species as the patient), is administered by injection or other suitable route one or more times per week until the animal's condition is improved, or it can be administered indefinitely.

Manufacture of Medicaments. The present invention also relates to the use of IMX129840 cytokine polypeptides, fragments, and variants; nucleic acids encoding the IMX129840 cytokine family polypeptides, fragments, and variants; agonists or antagonists of the IMX129840 cytokine polypeptides such as antibodies; IMX129840 cytokine polypeptide binding partners; complexes formed from the IMX129840 cytokine family polypeptides, fragments, variants, and binding partners, etc, in the manufacture of a medicament for the prevention or therapeutic treatment of each medical disorder disclosed herein.

### EXAMPLES

The following examples are intended to illustrate particular embodiments and not to limit the scope of the invention.

### Example 1

### Identification of IMX129840-1, -2, -3 and -4, New Members of the Human Cytokine Family

A data set was received from Celera Genomics (Rockville, Md.) containing a listing of amino acid sequences predicted, using automated approaches such as the GENSCAN program (Miyajima et al., 2000, Biochem Biophys Res Commun 272: 801-807), to be encoded by the human genome. These amino acid sequence predictions were analyzed using GeneFold (Tripos, Inc., St. Louis, Mo.; Jaroszewski et al., 1998, Prot Sci 7: 1431-1440), a protein threading program that overlays a query protein sequence onto structural representatives of the Protein Data Bank (PDB) (Berman et al., 2000, Nucleic Acids Res 28: 235-242). As described above, four alpha helix bundle (4AHB) cytokine family members are characterized by a particular three-dimensional structure; this four-helical structure can be predicted from their primary amino acid sequences by using protein-threading algorithms such as GeneFold. To use GeneFold to classify new members of a protein family, the new protein sequence is entered into the program, which assigns a probability score that reflects how well it folds onto known protein structures (“template” structures) that are present in the GeneFold database. For scoring, GeneFold relies on primary amino acid sequence similarity, burial patterns of residues, local interactions, and secondary structure comparisons. The GeneFold program folds (or threads) the amino acid sequence onto all of the template structures in a database of protein folds, which includes the solved structures for several human cytokine/growth factor polypeptides such as Granulocyte-Macrophage Colony-Stimulating Factor (GM-CSF), Granulocyte Colony-Stimulating Factor (G-CSF), and interferon-alpha 2 (IFN-alpha2). For each comparison, three different scores are calculated, based on (i) sequence only; (ii) sequence plus local conformation preferences plus burial terms; and (iii) sequence plus local conformation preferences plus burial terms plus secondary structure. In each instance, the program determines the optimal alignment, calculates the probability (P-value) that this degree of alignment occurred by chance, and reports the inverse of the P-value as the score. These scores therefore reflect the degree to which the new protein matches the various reference structures and are useful for assigning a new protein to membership in a known family of proteins. When one of the polypeptides predicted from the human genome data was threaded into the GeneFold program, several of the highest-scoring template structures for this polypeptide were cytokine or growth factor templates, although structural similarities to other alpha-helix containing proteins were also identified. This predicted polypeptide sequence was then used to identify the human genome sequences that encode it, and the IMX129840-1 polypeptide sequence was determined by analysis of these predicted exons. The genomic contig containing the IMX129840-1 coding sequences, GenBank accession number AC011445.6 (“the AC011445 contig”), was analyzed and three additional potential coding sequences for 4AHB cytokines were identified on that same contig and were named IMX129840-2, IMX129840-3, and IMX129840-4.

The predicted exon sequences of IMX129840-1 were used to design oligonucleotide primers and isolate an IMX129840-1 cDNA molecule (SEQ ID NO:1) encoding a IMX129840 cytokine polypeptide having the amino acid sequence shown in SEQ ID NO:2; nucleotides 58 through 657 of SEQ ID NO:1 encode SEQ ID NO:2, with nucleotides 658 through 660 corresponding to a stop codon and nucleotides 796 through 801 likely representing a polyadenylation signal for the poly(A) tail at nucleotides 816 (or 817 or 818) through 838. The IMX129840-2 cytokine coding sequence (nucleotides 141 through 740 of SEQ ID NO:3) encodes the polypeptide sequence presented as SEQ ID NO:4, with nucleotides 741 through 743 corresponding to a stop codon. The IMX129840-3 cytokine coding sequence (nucleotides 141 through 740 of SEQ ID NO:5) encodes the polypeptide sequence presented as SEQ ID NO:6, with nucleotides 741 through 743 corresponding to a stop codon. The IMX129840-3 predicted exon and cytokine polypeptide sequences (SEQ ID NOs 5 and 6) are extremely similar but not identical to those of IMX129840-2 (SEQ ID NOs 3 and 4), and are located within approximately 25 kb of each other and in the opposite orientation to each other on the AC011445 contig, consistent with an evolutionarily recent gene duplication event. The AC011445 contig also contains a fourth potential cytokine coding sequence, IMX129840-4, that has regions of significant primary amino acid sequence similarity to IMX129840-1, -2, and -3. This tightly clustered arrangement of apparently duplicated genes, having extremely similar exon/intron configurations, is very similar to that of the short-chain 4AHB cytokines GM-CSF, IL-3, and IL-5, which are clustered together on human chromosome 5.

The predicted exons for IMX129840-1 and -2 were confirmed by the isolation of a full-length cDNA clones (SEQ ID NO:1 and SEQ ID NO:3, respectively), and PCR amplification experiments have confirmed that cDNAs with structures consistent with those predicted for IMX129840-2 and -3 are present in cDNA libraries made from tissues such as testes. In addition, the regions of the human genome encoding IMX129840-1, IMX129840-2, and IMX129840-4 were compared with mouse chromosome 7 genomic sequences to compare the degree of evolutionary conservation of predicted IMX129840 exon and intron regions. A good level of inter-species sequence conservation was observed between the predicted IMX129840-1 and -2 exons and the mouse genome sequences, and significantly less similarity was observed in the intron regions, consistent with the predicted exon sequences having an evolutionarily conserved function in encoding IMX129840 cytokine polypeptides. The comparison of the human and murine genomic sequences identified region containing a potential IMX129840-4 exon at approximately nucleotides 14580-14850 of the AC011445 contig (see the table below); this region includes a potential open frame (presented as SEQ ID NO:7) encoding twenty or more amino acids (presented as SEQ ID NO:8). The five-nucleotide stretch from position 247 through 251 of SEQ ID NO:7 has been determined to be “CAAGG”, but it is noted that an insertion of one nucleotide into this region will generate a reading frame and a predicted amino acid sequence that is more similar to the IMX129840-1, -2, and -3 cytokine polypeptides.

Murine cytokine coding sequences and polypeptides corresponding to human IMX129840 cytokine sequences are presented herein: partial coding and amino acid sequences for murine IMX129840-1 (SEQ ID NOs 9 and 10, respectively) and complete coding and amino acid sequences for murine IMX129840-2 (SEQ ID NOs 11 and 12, respectively). Nucleotides 292 through 897 of SEQ ID NO:11 encodes the murine IMX129840-2 cytokine polypeptide sequence presented as SEQ ID NO:12, with nucleotides 898 through 900 of SEQ ID NO:11 corresponding to a stop codon.

The human IMX129840-1, -2, -3, and -4 cytokine coding sequences were compared with publicly available preliminary human genomic DNA sequences, and the following chromosome 19 contigs were identified as containing IMX129840 cytokine coding sequences: AC011445.6 (IMX129840-1, -2, -3, and -4) and AC018477.12 (IMX129840-1 and -4). The approximate positions of the exons containing IMX129840 cytokine coding sequence in the AC011445.6 contig are shown in the table below, along with their locations relative to SEQ ID NOs 1, 3, and 5; note that the 5′ and 3′ untranslated regions may extend further along the contig sequence beyond those portions that correspond to SEQ ID NOs 1, 3, and 5, as indicated by the parentheses around the AC011445.6 endpoints in the table. Note that additional 3′ UTR sequences may be predicted for IMX129840-3 based on significant sequence similarity to the 3′ UTR region of IMX129840-2; this suggests both that IMX129840-3 may have an additional intron within the genomic sequence that encodes its 3′ UTR, and that there may be functional sequences, such as elements regulating mRNA stability and degradation, in the 3′ UTRs of these cytokine genes as there are in the 3′ UTRs of the human GMCSF, GROalpha, IL-1 beta, and IL-3 genes. Also, a perfect duplication of at least a portion of the IMX129840-3 coding region is present at approximately nucleotides 135212-138020 of the AC011445 contig in the opposite orientation to IMX129840-3; however, this additional, perfectly duplicated sequence is not present in an updated assembly of overlapping contigs in this region (see GenBank accession number NT_{—}011260), and is believed to be an artifact of the assembly of the AC011445 contig sequence. Corresponding positions of IMX129840 cytokine gene exons in human contig AC011445.6 and in cDNA sequences:

| | | Position in SEQ ID |
|---|---|---|
| IMX129840-1 Exons | Position in AC011445.6 | NO: 1 |
| Exon 1 | (77467)-77695 | 1-228 |
| Exon 2 | 77907-77984 | 229-306 |
| Exon 3 | 79066-79209 | 307-450 |
| Exon 4 | 79315-79398 | 451-534 |
| Exon 5 | 79493-(79775) | 535-817 |
| | | Position in SEQ ID |
| IMX129840-2 Exons | Position in AC011445.6 | NO: 3 |
| Exon 1 | (26,319)-26,170 | 1-150 |
| Exon 2 | 26,071-25,890 | 151-332 |
| Exon 3 | 25,596-25,519 | 333-410 |
| Exon 4 | 25,259-25,110 | 411-560 |
| Exon 5 | 25,009-24,926 | 561-644 |
| Exon 6 | 24,832-(24,389) | 645-1088 |
| | | Position in SEQ ID |
| IMX129840-3 Exons | Position in AC011445.6 | NO: 5 |
| Exon 1 | (49531)-49680 | 1-150 |
| Exon 2 | 49779-49960 | 151-332 |
| Exon 3 | 50254-50331 | 333-410 |
| Exon 4 | 50590-50739 | 411-560 |
| Exon 5 | 50840-50923 | 561-644 |
| Exon 6 | 51017-51200 | 645-828 |
| (continuation of Exon | (51201-ca. 51360) | (based |
| 6) | | on SEQ ID NO: 3) |
| (possible Exon 7) | (ca. 52485-ca. 52580) | (based |
| | | on SEQ ID NO: 3) |

| Possible IMX129840-4 Exons | Approx. Position in AC011445.6 |
|---|---|
| (Exon 1) | ca. 18900-ca. 18300 |
| (Exon 2) | ca. 18050-ca. 17900 |
| (Exon 3) | ca. 17800-ca. 17700 |
| (Exon 4) | ca. 17450-ca. 17380 |
| (Exon 5) | ca. 17160-ca. 17100 |
| (Exon 6) | ca. 16670-ca. 16400 |
| (Exon 7) | ca. 14580-ca. 14850 |
| (Exon 8) | ca. 14360-ca. 14290 |
| (Exon 9) | ca. 13270-ca. 13230 |
| (Exon 10) | ca. 12530-ca. 12500 |

The genomic sequences comprising human IMX129840 cytokine exons map to the 19q13.2 region of human chromosome 19. Human IMX129840 nucleic acids such as SEQ ID NOs 1, 3, and 5 and fragments thereof are useful for the cytological identification of this chromosomal region, and for the genomic mapping of human heritable disorders such as the following disorders that have been genetically mapped to this region: Optic Atrophy Type 3; Progressive Heart Block Type 1; Spastic Paraplegia gene 12; and Orofacial Cleft gene 3. Murine genomic sequences for the murine IMX129840-1, -2, and -3 polypeptides are present on the GenBank AC073768.1 contig. The partial murine IMX129840-1 coding sequence of SEQ ID NO: 9 maps (in reverse orientation) to nucleotides 186880 through 187011 of GenBank AC073768.1. In a fashion similar to that of human IMX129840-2, murine IMX129840-2 genomic coding sequences are organized in 6 exons corresponding to SEQ ID NO:11, these exons are distributed throughout a region of AC073768.1 beginning at nucleotide 112877 and ending at nucleotide 114822. Six exons appearing to encode murine IMX129840-3 are present in reverse orientation in the region of AC073768.1 beginning at nucleotide 240303 and ending at nucleotide 242236.

Additional variations of IMX129840 cytokine polypeptides are provided, including variations between polypeptide isoforms, such as the IMX129840-2 and -3 polypeptides, and naturally occurring genomic variants of the IMX129840 cytokine sequences disclosed herein. Such variations may be incorporated into a IMX129840 cytokine polypeptide or nucleic acid individually or in any combination, or in combination with alternative splice variations. As one example, amino acid 10 of IMX129840-2 (SEQ ID NO:4) differs from amino acid 10 of IMX129840-3 (SEQ ID NO:6), where the change from a Met residue to a Thr residue was apparently caused by a single change from ‘T’ at position 169 of SEQ ID NO:3 to ‘C’ at the same position in SEQ ID NO:5, or vice versa. This variation and others are listed in the table below:

| Isoform Amino | | | |
|---|---|---|---|
| Acid Change | | | |
| (IMX129840-2 -> | Position in SEQ ID | Nucleotide | Position in SEQ ID |
| IMX129840-3) | NOs 4 and 6 | Change | NOs 3 and 5 |
| Silent (Thr) | 6 | C -> T | 158 |
| Met -> Thr | 10 | T -> C | 169 |
| Arg -> His | 32 | G -> A | 235 |
| Lys -> Arg | 74 | A -> G | 361 |
| Arg -> His | 76 | G -> A | 367 |
| Val -> Met | 96 | G -> A | 426 |
| Gly -> Val | 120 | G -> T | 499 |
| Silent (Asp) | 121 | T -> C | 503 |
| Leu -> Phe | 137 | C -> T | 549 |
| His -> Tyr | 160 | C -> T | 618 |
| Silent (Ser) | 195 | C -> T | 725 |
| n/a | 3′ UTR | T -> C | 748 |
| n/a | 3′ UTR | G -> A | 751 |
| n/a | 3′ UTR | G -> T | 795 |
| n/a | 3′ UTR | T -> C | 808 |
| Allelic Variant | | | |
| Amino Acid | | | |
| Change | Position in SEQ ID | Nucleotide | Position in SEQ ID |
| (IMX129840-1) | NO: 2 | Change | NO: 1 |
| Asp -> Gly | 37 | A -> G | 167 |
| Asn -> Asp | 188 | A -> G | 619 |
| n/a | 3′ UTR | G -> A | 793 |

The amino acid sequences of the IMX129840 cytokine polypeptides (SEQ ID NOs 2, 4, and 6) were compared with each other using the GCG “pretty” multiple sequence alignment program, with amino acid similarity scoring matrix=blosum62, gap creation penalty=8, and gap extension penalty=2. An alignment of these sequences is shown in Table 1, and includes consensus residues which are identical among all three of the amino acid sequences in the alignment. The capitalized residues in the alignment are those which match the consensus residues. The numbering of amino acid residues in Table 1 corresponds to the position of those residues in the IMX129840-2 and -3 cytokine amino acid sequences (SEQ ID NOs 4 and 6).

Amino acid substitutions and other alterations (deletions, insertions, etc.) to IMX129840 cytokine amino acid sequences (e.g. SEQ ID NOs 2, 4, and 6) are predicted to be more likely to alter or disrupt IMX129840 cytokine polypeptide activities if they result in changes to the capitalized residues of the amino acid sequences as shown in Table, and particularly if those changes do not substitute an amino acid of similar chemical properties (such as substitution of any one of the aliphatic residues—Ala, Gly, Leu, Ile, or Val—for another aliphatic residue), or a residue present in other cytokine polypeptides at that conserved position. Conversely, if a change is made to an IMX129840 cytokine amino acid sequence resulting in substitution of the residue at that position in the alignment from one of the other Table 1 cytokine polypeptide sequences, it is less likely that such an alteration will affect the function of the altered IMX129840 cytokine polypeptide. For example, the consensus residue at position 48 in Table 1 is serine; substitution of the chemically similar threonine for serine at that position is less likely to alter the function of the polypeptide than substitution of tryptophan or tyrosine etc. Embodiments of the invention include IMX129840 cytokine polypeptides and fragments of IMX129840 cytokine polypeptides, comprising altered amino acid sequences. Altered IMX129840 cytokine polypeptide sequences share at least 30%, or more preferably at least 40%, or more preferably at least 50%, or more preferably at least 55%, or more preferably at least 60%, or more preferably at least 65%, or more preferably at least 70%, or more preferably at least 75%, or more preferably at least 80%, or more preferably at least 85%, or more preferably at least 90%, or more preferably at least 95%, or more preferably at least 97.5%, or more preferably at least 99%, or most preferably at least 99.5% amino acid identity with one or more of the cytokine amino acid sequences shown in Table 1. When IMX129840 cytokine polypeptide variants according to the invention, such as allelic variants or IMX129840 cytokine polypeptides having deliberately engineered modifications, are analyzed using GeneFold as described further herein, at least one of the ten top-scoring template structures within one of the three types of GeneFold scoring methods will be cytokine or growth factor polypeptides. The score for the top-scoring cytokine or growth factor template structures, using any of the three types of score reported by GeneFold (sequence only, sequence plus local conformation preferences plus burial terms, or sequence plus local conformation preferences plus burial terms plus secondary structure) preferably will be at least 20, more preferably at least 30, more preferably at least 40, still more preferably at least 50, and most preferably at least 60.

TABLE 1

Alignment of IMX129840-1, -2, and -3 cytokine
amino acid sequences

| | |
|---|---|
| | 1 50 |
| IMX129840-2 | mkldMtgdcm pVLVlmaavL tVtGaVPvar lrgalpdarG CHIagFKSLS |
| IMX129840-3 | mkldMtgdct pVLVlmaavL tVtGaVPvar lhgalpdarG CHIagFKSLS |
| IMX129840-1 | ˜˜˜˜Maaawt vVLVtlvlgL aVaGpVPtsk ...ptttgkG CHIdrFKSLS |
| | ▪ ▪ ▪ ▪ ▪ ▪ ▪ ▪ ▪ ▪ ▪ ▪ ▪ ▪ ▪ ▪ ▪ ▪ |
| consensus | ----M----- -VLV-----L -V-G-VP--- ---------G CHI--FKSLS |
| | 51 100 |
| IMX129840-2 | PQELgaFKrA kDALEESLlL KdckCrSrlF PrtWDLRgLQ VRERPvALEA |
| IMX129840-3 | PQELgaFKrA kDALEESLlL KdcrChSrlF PrtWDLRgLQ VRERPmALEA |
| IMX129840-1 | PQELasFKkA rDALEESLkL KnwsCsSpvF PgnWDLRlLQ VRERPvALEA |
| consensus | PQEL--FK-A -DALEESL-L K---C-S--F P--WDLR-LQ VRERP-ALEA |
| | 101 150 |
| IMX129840-2 | ELALTLKVLE AtAdtdPALg DVLDQPLHTL HHILSQlrAC IQPQPTAGPR |
| IMX129840-3 | ELALTLKVLE AtAdtdPALv DVLDQPLHTL HHILSQfrAC IQPQPTAGPR |
| IMX129840-1 | ELALTLKVLE AaA..gPALe DVLDQPLHTL HHILSQlgAC IQPQPTAGPR |
| consensus | ELALTLKVLE A-A---PAL- DVLDQPLHTL HHILSQ--AC IQPQPTAGPR |
| | 151 200 |
| IMX129840-2 | tRGRLHHWLh RLQEAPKKES pGCLEASVTF NLFRLLTRDL ncVAsGdLCv |
| IMX129840-3 | tRGRLHHWLy RLQEAPKKES pGCLEASVTF NLFRLLTRDL ncVAsGdLCv |
| IMX129840-1 | pRGRLHHWLh RLQEAPKKES aGCLEASVTF NLFRLLTRDL kyVAdGnLCl |
| consensus | -RGRLHHWL- RLQEAPKKES -GCLEASVTF NLFRLLTRDL --VA-G-LC- |
| | 201 |
| IMX129840-2 | ˜˜˜˜˜˜˜˜˜ |
| IMX129840-3 | ˜˜˜˜˜˜˜˜˜ |
| IMX129840-1 | rtsthpest |
| consensus | ˜˜˜˜˜˜˜˜˜ |

▪▪▪: signal sequence

### Example 2

### Analysis of IMX129840 Cytokine Expression by Real-Time Quantitative PCR

RNA samples were obtained from a variety of tissue sources and from cells or tissues treated with a variety of compounds; these RNA samples included commercially available RNA (Ambion, Austin, Tex.; Clontech Laboratories, Palo Alto, Calif.; and Stratagene, La Jolla, Calif.). The RNA samples were DNase treated (part # 1906, Ambion, Austin, Tex.), and reverse transcribed into a population of cDNA molecules using TaqMan Reverse Transcription Reagents (part # N808-0234, Applied Biosystems, Foster City, Calif.) according to the manufacturer's instructions using random hexamers. Each population of cDNA molecules was placed into specific wells of a multi-well plate at either 5 ng or 20 ng per well and run in triplicate. Pooling was used when same tissue types and stimulation conditions were applied but collected from different donors. Negative control wells were included in each multi-well plate of samples.

Sets of probes and oligonucleotide primers complementary to mRNAs encoding human IMX129840-1 (SEQ ID NO:2) polypeptides and to mRNAs encoding human IMX129840-2 (SEQ ID NO:4) polypeptides were designed using Primer Express software (Applied Biosystems, Foster City, Calif.) and synthesized, and PCR conditions for these probe/primer sets were optimized to produce a steady and logarithmic increase in PCR product every thermal cycle between approximately cycle 20 and cycle 36. The forward IMX129840-1 primer used was 5′ AGG CCC TGT CCC CAC TTC 3′ (SEQ ID NO:13); the reverse IMX129840-1 primer used was 5′ GAG ATT TGA ACC TGC CAA TGT G 3′ (SEQ ID NO:14); and the labeled probe used for human IMX129840-1 was 5′ CCC ACC ACA ACT GGG AAG GGC TG 3′ (SEQ ID NO:15). The forward IMX129840-2 primer used was 5′ GGA GCT GCA GGC CTT TAA GA 3′ (SEQ ID NO:16); the reverse IMX129840-2 primer used was 5′ GCG GCA CTT GCA GTC CTT 3′ (SEQ ID NO:17); and the labeled probe used for human IMX129840-2 was 5′ CCA AAG ATG CCT TAG AAG AGT CGC TTC TGC T 3′ (SEQ ID NO:18). Oligonucleotide primer sets complementary to 18S RNA and to mRNAs encoding certain ‘housekeeper’ proteins—beta-actin, HPRT (hypoxanthine phosphoribosyltransferase), DHFR (dihydrofolate reductase), PKG (phosphoglycerate kinase), and GAPDH (glyceraldehyde-3-phosphate dehydrogenase)—were synthesized and PCR conditions were optimized for these primer sets also. Multiplex TAQMAN PCR reactions using both human IMX129840-1 and beta-actin probe/primer sets, or both human IMX129840-2 and GAPDH probe/primer sets, were set up in 25-microliter volumes with TAQMAN Universal PCR Master Mix (part # 4304437, Applied Biosystems, Foster City, Calif.) on an Applied Biosystems Prism 7700 Sequence Detection System. Threshold cycle values (C_{T}) were determined using Sequence Detector software version 1.7a (Applied Biosystems, Foster City, Calif.), and delta C_{T} was calculated and transformed to 2E(-dC_{T}), which is 2 to the minus delta C_{T}, for relative expression comparison of IMX129840-1 to beta-actin or IMX129840-2 to GAPDH.

Expression of human IMX129840-1 relative to beta-actin expression, and of human IMX129840-2 relative to GAPDH expression, was analyzed in a variety of adult and fetal RNA samples. This analysis indicated that human IMX129840-1 and -2 messages are detectable and less abundant than housekeeper mRNAs in certain adult and fetal tissues, such as adult and fetal brain or adult testis (see below); a ratio of 0.00140683 indicates that the expression of human IMX129840-1 in this adult brain sample is about 0.14% of that of beta-actin. Note that in the tables below, a ratio of ‘0.0’ indicates that the Avg. CT value for the IMX12984 expression was too high to meet the criterion for detection, which is set at CT values of 36 or lower.

| | IMX129840-1 | beta-actin | IMX129840-1: | Minimum | Maximum |
|---|---|---|---|---|---|
| Sample | Avg CT | Avg CT | beta-actin Ratio | (Minus Err) | (Plus Err) |
| Adult Brain | 34.66 | 25.1867 | 0.00140683 | 0.00116114 | 0.00170451 |
| Fetal Brain | 34.2033 | 24.2167 | 0.00098563 | 0.00087105 | 0.00111528 |
| | IMX129840-2 | GAPDH | IMX129840-2: | Minimum | Maximum |
| Sample | Avg CT | Avg CT | GAPDH Ratio | (Minus Err) | (Plus Err) |
| Adult Testis | 34.85 | 23.3367 | 0.00034209 | 0.00029398 | 0.00039807 |

Analysis of human IMX129840-1 and -2 expression relative to housekeeper gene expression in additional RNA samples indicated that there was some detectable expression of in human IMX129840-1 in human osteoblasts (MG-63) cultured for 7 days in the absence or presence of vitamin D (see below). Human IMX129840-1 and -2 expression was detected in real-time quantitative PCR experiments with RNA samples from adult liver hepatocytes, either unstimulated, or treated with 1 microgram/ml LPS for 2 hours, or treated with 100 microgram/ml each of IL-1, IL-18, and TNF for 24 hours. The results of these real-time quantitative PCR experiments are shown in the table below. These data indicate that in the untreated and cytokine-treated hepatocytes, human IMX129840-1 was expressed at a low level relative to beta-actin expression. Treatment with IL-1, IL-18, and TNF cytokines did not significantly change the level of human IMX129840-1 expression relative to beta-actin expression. Expression of IMX129840-2 was generally somewhat higher relative to GAPDH expression in these hepatocytes, and treatment with LPS or with the combination of IL-1, IL-18, and TNF cytokines reduced human IMX129840-2 expression slightly (i.e. to approximately 80% of its levels in untreated cells).

| | IMX129840-1 | beta-actin | IMX129840-1: | Minimum | Maximum |
|---|---|---|---|---|---|
| Sample | Avg CT | Avg CT | beta-actin Ratio | (Minus Err) | (Plus Err) |
| Osteoblast day 7 | 35.8767 | 19.63 | 0.00001286 | 0.00000799 | 0.0000207 |
| Osteoblast day 7 vit. D | 35.0433 | 18.5767 | 0.00001104 | 0.00000726 | 0.00001679 |
| Liver (no stim.) | 35.9767 | 18.1933 | 0.00000443 | 0.0000033 | 0.00000596 |
| Liver LPS | 37.7267 | 18.0167 | 0.0 | 0.0 | 0.0 |
| Liver IL1/IL18/TNF | 35.83 | 18.28 | 0.0000052 | 0.00000385 | 0.00000705 |
| | IMX129840-2 | GAPDH | IMX129840-2: | Minimum | Maximum |
| Sample | Avg CT | Avg CT | GAPDH Ratio | (Minus Err) | (Plus Err) |
| Liver (no stim.) | 34.8633 | 21.3567 | 0.00008592 | 0.00007299 | 0.00010114 |
| Liver LPS | 35.0767 | 21.19 | 0.00006602 | 0.00005965 | 0.00007308 |
| Liver IL1/IL18/TNF | 34.9067 | 21.0433 | 0.0000671 | 0.0000609 | 0.00007393 |

Further real-time quantitative PCR experiments indicated that in certain cell types, treatment with interferon-gamma (IFNg), other cytokines, or UV radiation significantly altered expression of human IMX129840-1 and -2 relative to housekeeper gene expression (see the table below). For expression of human IMX129840-1 relative to beta-actin expression, the following observations can be made based on the data in the table below. IMX129840-1 expression was detected in a population of regulatory Tcells (CD4+CD25+). Some treatments tend to reduce IMX129840-1 expression: anti-CD3 antibody treatment of peripheral blood mononuclear cells (PBMC); IL-15 and possibly IL-12 treatment of natural killer (NK) cells; treatment of skin keratinocytes (HaCaT) with LPS; possibly, treatment of B cells with a mixture including CD40 ligand (CD40L) and IL-4; and in the lung epithelial adenocarcinoma cell line Calu3, treatment with a mixture of IL-4 and IL-13. Interestingly, treatment of the colon epithelial carcinoma line T84 with IL-4 and IL-13 has the opposite effect to that in Calu3 cells: in T84 cells, these cytokines increase the expression of human IMX129840-1 relative to beta-actin. Additional treatments that increase expression of human IMX129840-1 relative to beta-actin include treating HaCaT cells with ultraviolet radiation (UV); treating T84, HaCaT, or Calu3 cells with mixtures of IL-1, TNF, and IL-18 cytokines; and most strikingly, treating T84, HaCaT, or Calu3 cells with interferon-gamma (IFNg). In the Calu3 cells, treatment with IFNg increases expression of human IMX129840-1 relative to beta-actin approximately 40-fold, with the result that human IMX129840-1 is expressed at almost twice the level of the beta-actin housekeeping gene in these treated cells. The treatment of Calu3 cells with the IL-4/IL-13 combination reduces relative human IMX129840-1 expression (as mentioned above) while treatment of these cells with IFNg greatly increases its relative expression. These effects of IL-4/IL-13 and IFNg in Calu3 cells are consistent with the contrasting effects IL-4/IL-13 and IFNg have on the ability of Calu3 cells to maintain lung epithelial barrier function and to repair wounds (Ahdieh et al., 2001, Am J Physiol Cell Physiol 281: C2029-2038). Expression of human IMX129840-2 relative to GAPDH was also increased in T84, HaCaT, and Calu3 cells by IFNg treatment, and treatment with a mixture of IL-1, TNF, and IL-18 cytokines also increased relative human IMX129840-2 expression in Calu3 cells. The increase in human IMX129840-1 and -2 expression in these epithelial cell types in response to IFNg treatment suggests that the IMX129840-1 and -2 cytokines play a role in epithelial cell functions and/or disorders affected by IFNg, such as psoriasis and the regulation of barrier function in lung and intestinal epithelia.

| | IMX129840-1 | beta-actin | IMX129840-1: | Minimum | Maximum |
|---|---|---|---|---|---|
| Sample | Avg CT | Avg CT | beta-actin Ratio | (Minus Err) | (Plus Err) |
| Regulatory Tcells | 35.9967 | 20.48 | 2.133E−05 | 1.904E−05 | 2.39E−05 |
| PBMC no stim | 34.3733 | 17.827 | 1.045E−05 | 8.47E−06 | 1.289E−05 |
| PBMC aCD3 | 35.7333 | 17.233 | 2.7E−06 | 2.39E−06 | 3.05E−06 |
| T84 no stim | 35.1967 | 19.57 | 1.977E−05 | 1.588E−05 | 2.461E−05 |
| T84 IL1/IL18/TNFa | 34.1633 | 19.287 | 3.324E−05 | 3.113E−05 | 3.55E−05 |
| T84 IL4/IL13 | 34.04 | 19.873 | 5.438E−05 | 5.009E−05 | 5.903E−05 |
| T84 IFNg | 31.14 | 19.347 | 2.817E−04 | 2.484E−04 | 3.196E−04 |
| NHBE no stim | 37.8433 | 18.96 | 0 | 0 | 0 |
| NHBE IFNg | 34.48 | 19.357 | 2.802E−05 | 2.584E−05 | 3.038E−05 |
| NK no stim | 32.9 | 18.197 | 3.748E−05 | 3.373E−05 | 4.166E−05 |
| NK IL12 | 36.74 | 17.487 | 0 | 0 | 0 |
| NK IL15 | 35.1733 | 16.44 | 2.29E−06 | 2.08E−06 | 2.53E−06 |
| B cell no stim | 35.3667 | 19.623 | 1.823E−05 | 1.694E−05 | 1.962E−05 |
| B cell SAC/CD40L/IL4 | 38.3 | 16.673 | 0 | 0 | 0 |
| HaCaT no stim 5.25 h | 36.2767 | 17.377 | 0 | 0 | 0 |
| HaCaT no stim 18 h | 34.8067 | 17.323 | 5.46E−06 | 4.4E−06 | 6.78E−06 |
| HaCaT LPS 5.25 h | 36.0433 | 17.997 | 0 | 0 | 0 |
| HaCaT LPS 18 h | 35.8167 | 17.447 | 2.95E−06 | 2.09E−06 | 4.16E−06 |
| HaCaT IL1/TNF/IL18 | 35.3433 | 17.613 | 4.6E−06 | 3.68E−06 | 5.76E−06 |
| 5.25 h | | | | | |
| HaCaT IL1/TNF/IL18 18 h | 34.86 | 18.09 | 8.95E−06 | 7.5E−06 | 1.068E−05 |
| HaCaT UV 0 s 18 h | 35.11 | 17.48 | 4.93E−06 | 3.33E−06 | 7.3E−06 |
| HaCaT UV 5 s 5.25 h | 33.5033 | 17.82 | 1.9E−05 | 1.614E−05 | 2.238E−05 |
| HaCaT IFNg 5.25 h | 33.3667 | 17.66 | 1.87E−05 | 1.585E−05 | 2.206E−05 |
| HaCaT IFNg 18 h | 31.83 | 17.41 | 4.562E−05 | 4.134E−05 | 5.034E−05 |
| Calu3 no stim | 34.2867 | 29.72 | 0.0421984 | 0.0368101 | 0.0483756 |
| Calu3 IL4/IL13 | 35.4533 | 29.717 | 0.0187539 | 0.0157184 | 0.0223755 |
| Calu3 IL1/IL18/TNFa | 31.6933 | 29.547 | 0.2258338 | 0.1939271 | 0.2629902 |
| Calu3 IFNg | 31.3367 | 32.253 | 1.8877486 | 0.7378551 | 4.8296677 |

| | IMX129840-2 | GAPDH | IMX129840-2: | Minimum | Maximum |
|---|---|---|---|---|---|
| Sample | Avg CT | Avg CT | GAPDH Ratio | (Minus Err) | (Plus Err) |
| T84 no stim | 37.09 | 20.527 | 0 | 0 | 0 |
| T84 IFNg | 33.49 | 21.04 | 0.0001787 | 0.0001627 | 0.0001963 |
| HaCaT no stim 18 h | 37.92 | 18.89 | 0 | 0 | 0 |
| HaCaT IFNg 18 h | 34.3867 | 18.713 | 1.914E−05 | 1.687E−05 | 0.0000217 |
| Calu3 no stim | 35.33 | 22.907 | 0.0001821 | 0.0001405 | 0.0002359 |
| Calu3 IL1/IL18/TNFa | 31.26 | 22.847 | 0.0029332 | 0.0028041 | 0.0030681 |
| Calu3 IFNg | 32.05 | 23.557 | 0.0027749 | 0.0024755 | 0.0031106 |

### Example 3

### Monoclonal Antibodies That Bind Polypeptides of the Invention

This example illustrates a method for preparing monoclonal antibodies that bind IMX129840 cytokine polypeptides. Other conventional techniques may be used, such as those described in U.S. Pat. No. 4,411,993. Suitable immunogens that may be employed in generating such antibodies include, but are not limited to, purified IMX129840 cytokine polypeptide, an immunogenic fragment thereof, and cells expressing high levels of IMX129840 cytokine polypeptide or an immunogenic fragment thereof. DNA encoding a IMX129840 cytokine polypeptide can also be used as an immunogen, for example, as reviewed by Pardoll and Beckerleg in Immunity 3: 165, 1995.

Rodents (BALB/c mice or Lewis rats, for example) are immunized with IMX129840 cytokine polypeptide immunogen emulsified in an adjuvant (such as complete or incomplete Freund's adjuvant, alum, or another adjuvant, such as Ribi adjuvant R700 (Ribi, Hamilton, Mont.)), and injected in amounts ranging from 10-100 micrograms subcutaneously or intraperitoneally. DNA may be given intradermally (Raz et al., 1994, Proc. Natl. Acad. Sci. USA 91: 9519) or intamuscularly (Wang et al., 1993, Proc. Natl. Acad. Sci. USA 90: 4156); saline has been found to be a suitable diluent for DNA-based antigens. Ten days to three weeks days later, the immunized animals are boosted with additional immunogen and periodically boosted thereafter on a weekly, biweekly or every third week immunization schedule.

Serum samples are periodically taken by retro-orbital bleeding or tail-tip excision to test for IMX129840 cytokine polypeptide-specific antibodies by dot-blot assay, ELISA (enzyme-linked immunosorbent assay), immunoprecipitation, or other suitable assays, such as FACS analysis of inhibition of binding of IMX129840 cytokine polypeptide to a IMX129840 cytokine polypeptide binding partner. Following detection of an appropriate antibody titer, positive animals are provided one last intravenous injection of IMX129840 cytokine polypeptide in saline. Three to four days later, the animals are sacrificed, and spleen cells are harvested and fused to a murine myeloma cell line, e.g., NS1 or preferably P3X63Ag8.653 (ATCC CRL-1580). These cell fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells may be screened by ELISA for reactivity against purified IMX129840 cytokine polypeptide by adaptations of the techniques disclosed in Engvall et al., (Immunochem. 8: 871, 1971) and in U.S. Pat. No. 4,703,004. A preferred screening technique is the antibody capture technique described in Beckmann et al., (J. Immunol. 144: 4212, 1990). Positive hybridoma cells can be injected intraperitoneally into syngeneic rodents to produce ascites containing high concentrations (for example, greater than 1 milligram per milliliter) of anti-IMX129840 cytokine polypeptide monoclonal antibodies. Alternatively, hybridoma cells can be grown in vitro in flasks or roller bottles by various techniques. Monoclonal antibodies can be purified by ammonium sulfate precipitation, followed by gel exclusion chromatography. Alternatively, affinity chromatography based upon binding of antibody to protein A or protein G can also be used, as can affinity chromatography based upon binding to IMX129840 cytokine polypeptide.

### Example 4

### Antisense Inhibition of IMX129840 Cytokine Nucleic Acid Expression

In accordance with the present invention, a series of oligonucleotides are designed to target different regions of mRNA molecules encoding IMX129840 cytokines, using the nucleotide sequences of SEQ ID NOs 1, 3, 5, 7, 9, and 11 as the bases for the design of the oligonucleotides. Oligonucleotide sequences, such as pools of degenerate oligonucleotides, may be selected that will hybridize to mRNA molecules encoding all of IMX129840-1, -2, -3, and -4, or to mRNA molecules encoding a subset thereof; however, due to the extremely high degree of similarity between the IMX129840-2 and IMX129840-3 coding sequences, it would be difficult to design an oligonucleotide that will bind to the coding sequence of one but not the other. The oligonucleotides are selected to be approximately 10, 12, 15, 18, or more preferably 20 nucleotide residues in length, and to have a predicted hybridization temperature that is at least 37 degrees C. Preferably, the oligonucleotides are selected so that some will hybridize toward the 5′ region of the mRNA molecule, others will hybridize to the coding region, and still others will hybridize to the 3′ region of the mRNA molecule. Methods such as those of Gray and Clark (U.S. Pat. Nos. 5,856,103 and 6,183,966) can be used to select oligonucleotides that form the most stable hybrid structures with target sequences, as such oligonucleotides are desirable for use as antisense inhibitors.

The oligonucleotides may be oligodeoxynucleotides, with phosphorothioate backbones (internucleoside linkages) throughout, or may have a variety of different types of internucleoside linkages. Generally, methods for the preparation, purification, and use of a variety of chemically modified oligonucleotides are described in U.S. Pat. No. 5,948,680. As specific examples, the following types of nucleoside phosphoramidites may be used in oligonucleotide synthesis: deoxy and 2′-alkoxy amidites; 2′-fluoro amidites such as 2′-fluorodeoxyadenosine amidites, 2′-fluorodeoxyguanosine, 2′-fluorouridine, and 2′-fluorodeoxycytidine; 2′-O-(2-methoxyethyl)-modified amidites such as 2,2′-anhydro[1-(beta-D-arabino-furanosyl)-5-methyluridine], 2′-O-methoxyethyl-5-methyluridine, 2′-O-methoxyethyl-5′-O-dimethoxytrityl-5-methyluridine, 3′-O-acetyl-2′-O-methoxy-ethyl-5′-O-dimethoxytrityl-5-methyluridine, 3′-O-acetyl-2′-O-methoxyethyl-5′-O-dimethoxytrityl-5-methyl-4-triazoleuridine, 2′-O-methoxyethyl-5′-O-dimethoxytrityl-5-methylcytidine, N4-benzoyl-2′-O-methoxyethyl-5′-O-dimethoxytrityl-5-methylcytidine, and N4-benzoyl-2′-O-methoxyethyl-5′-O-di-methoxytrityl-5-methylcytidine-3′-amidite; 2′-O-(aminooxyethyl) nucleoside amidites and 2′-O-(dimethylaminooxyethyl) nucleoside amidites such as 2′-(dimethylaminooxyethoxy) nucleoside amidites, 5′-O-tert-butyldiphenylsilyl-O²-2′-anhydro-5-methyluridine, 5′-O-tert-butyl-diphenylsilyl-2′-O-(2-hydroxyethyl)-5-methyluridine, 2′-O-([2-phthalimidoxy)ethyl]-5′-t-butyldiphenyl-silyl-5-methyl-uridine, 5′-O-tert-butyldiphenylsilyl-2′-O-[(2-formadoximinooxy)ethyl]-5-methyluridine, 5′-O-tert-butyldiphenylsilyl-2′-O-[N,N-dimethylaminooxyethyl]-5-methyluridine, 2′-O-(dimethylaminooxy-ethyl)-5-methyluridine, 5′-O-DMT-2′-O-(dimethylaminooxyethyl)-5-methyluridine, and 5′-O-DMT-2′-O-(2-N,N-dimethylaminooxyethyl)-5-methyluridine-3′-[(2-cyanoethyl)-N,N-diisopropylphosphor-amidite]; and 2′-(aminooxyethoxy) nucleoside amidites such as N2-isobutyryl-6-O-diphenyl-carbamoyl-2′-O-(2-ethylacetyl)-5′-O-(4,4′-dimethoxytrityl)guanosine-3′-[(2-cyanoethyl)-N,N-diiso-propylphosphoramidite].

Modified oligonucleosides may also be used in oligonucleotide synthesis, for example methylenemethylimino-linked oligonucleosides, also called MMI-linked oligonucleosides; methylene-dimethylhydrazo-linked oligonucleosides, also called MDH-linked oligonucleosides; methylene-carbonylamino-linked oligonucleosides, also called amide-3-linked oligonucleosides; and methylene-aminocarbonyl-linked oligonucleosides, also called amide-4-linked oligonucleosides, as well as mixed backbone compounds having, for instance, alternating MMI and P═O or P═S linkages, which are prepared as described in U.S. Pat. Nos. 5,378,825, 5,386,023, 5,489,677, 5,602,240 and 5,610,289. Formacetal- and thioformacetal-linked oligonucleosides may also be used and are prepared as described in U.S. Pat. Nos. 5,264,562 and 5,264,564; and ethylene oxide linked oligonucleosides may also be used and are prepared as described in U.S. Pat. No. 5,223,618. Peptide nucleic acids (PNAs) may be used as in the same manner as the oligonucleotides described above, and are prepared in accordance with any of the various procedures referred to in Peptide Nucleic Acids (PNA): Synthesis, Properties and Potential Applications, Bioorganic & Medicinal Chemistry, 1996, 4, 5-23; and U.S. Pat. Nos. 5,539,082, 5,700,922, and 5,719,262.

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Some examples of different types of chimeric oligonucleotides are: [2′-O-Me]—[2′-deoxy]—[2′-O-Me] chimeric phosphorothioate oligonucleotides, [2′-O-(2-methoxyethyl)]—[2′-deoxy]—[2′-O-(methoxyethyl)] chimeric phosphorothioate oligonucleotides, and [2′-O-(2-methoxy-ethyl)phosphodiester]—[2′-deoxy phosphoro-thioate]—[2′-O-(2-methoxyethyl)phosphodiester] chimeric oligonucleotides, all of which may be prepared according to U.S. Pat. No. 5,948,680. In one preferred embodiment, chimeric oligonucleotides (“gapmers”) 18 nucleotides in length are utilized, composed of a central “gap” region consisting of ten 2′-deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. Other chimeric oligonucleotides, chimeric oligonucleosides, and mixed chimeric oligonucleotides/oligonucleosides are synthesized according to U.S. Pat. No. 5,623,065.

Oligonucleotides are preferably synthesized via solid phase P(III) phosphoramidite chemistry on an automated synthesizer capable of assembling 96 sequences simultaneously in a standard 96 well format. The concentration of oligonucleotide in each well is assessed by dilution of samples and UV absorption spectroscopy. The full-length integrity of the individual products is evaluated by capillary electrophoresis, and base and backbone composition is confirmed by mass analysis of the compounds utilizing electrospray-mass spectroscopy.

The effect of antisense compounds on target nucleic acid expression can be tested in any of a variety of cell types provided that the target nucleic acid is present at measurable levels. This can be routinely determined using, for example, PCR or Northern blot analysis. Cells are routinely maintained for up to 10 passages as recommended by the supplier. When cells reached 80% to 90% confluency, they are treated with oligonucleotide. For cells grown in 96-well plates, wells are washed once with 200 microliters OPTI-MEM-1 reduced-serum medium (Gibco BRL) and then treated with 130 microliters of OPTI-MEM-1 containing 3.75 g/mL LIPOFECTIN (Gibco BRL) and the desired oligonucleotide at a final concentration of 150 nM. After 4 hours of treatment, the medium is replaced with fresh medium. Cells are harvested 16 hours after oligonucleotide treatment. Preferably, the effect of several different oligonucleotides should be tested simultaneously, where the oligonucleotides hybridize to different portions of the target nucleic acid molecules, in order to identify the oligonucleotides producing the greatest degree of inhibition of expression of the target nucleic acid.

Antisense modulation of IMX129840 cytokine nucleic acid expression can be assayed in a variety of ways known in the art. For example, IMX129840 cytokine mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation and Northern blot analysis are taught in, for example, Ausubel, F. M. et al., Current Protocols in Molecular Biology, Volume 1, pp. 4.1.1-4.2.9 and 4.5.1-4.5.3, John Wiley & Sons, Inc., 1996. Real-time quantitative (PCR) can be conveniently accomplished using the commercially available ABI PRISM 7700 Sequence Detection System, available from PE-Applied Biosystems, Foster City, Calif. and used according to manufacturer's instructions. This fluorescence detection system allows high-throughput quantitation of PCR products. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or FAM, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples. Other methods of quantitative PCR analysis are also known in the art. IMX129840 cytokine protein levels can be quantitated in a variety of ways well known in the art, such as immunoprecipitation, Western blot analysis (immunoblotting), ELISA, or fluorescence-activated cell sorting (FACS). Antibodies directed to IMX129840 cytokine polypeptides can be prepared via conventional antibody generation methods such as those described herein. Immunoprecipitation methods, Western blot (immunoblot) analysis, and enzyme-linked immunosorbent assays (ELISA) are standard in the art (see, for example, Ausubel, F. M. et al., Current Protocols in Molecular Biology, Volume 2, pp. 10.16.1-10.16.11, 10.8.1-10.8.21, and 11.2.1-11.2.22, John Wiley & Sons, Inc., 1991).

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Sequences Presented in the Sequence Listing

| SEQ ID NO | Type | Description |
|---|---|---|
| SEQ ID NO: 1 | Nucleotide | Human IMX129840-1 cDNA sequence |
| SEQ ID NO: 2 | Amino acid | Human IMX129840-1 amino acid |
| | | sequence |
| SEQ ID NO: 3 | Nucleotide | Human IMX129840-2 predicted coding |
| | | sequence |
| SEQ ID NO: 4 | Amino acid | Human IMX129840-2 predicted amino |
| | | acid sequence |
| SEQ ID NO: 5 | Nucleotide | Human IMX129840-3 predicted coding |
| | | sequence |
| SEQ ID NO: 6 | Amino acid | Human IMX129840-3 predicted amino |
| | | acid sequence |
| SEQ ID NO: 7 | Nucleotide | Human IMX129840-4 predicted coding |
| | | sequence (partial) |
| SEQ ID NO: 8 | Amino acid | Human IMX129840-4 predicted amino |
| | | acid sequence (partial) |
| SEQ ID NO: 9 | Nucleotide | Mus musculus IMX129840-1 predicted |
| | | coding sequence (partial) |
| SEQ ID NO: 10 | Amino acid | Mus musculus IMX129840-1 predicted |
| | | amino acid sequence (partial) |
| SEQ ID NO: 11 | Nucleotide | Mus musculus IMX129840-2 predicted |
| | | coding sequence |
| SEQ ID NO: 12 | Amino acid | Mus musculus IMX129840-2 predicted |
| | | amino acid sequence |
| SEQ ID NO: 13 | Nucleotide | IMX129840-1 oligonucleotide primer |
| SEQ ID NO: 14 | Nucleotide | IMX129840-1 oligonucleotide primer |
| SEQ ID NO: 15 | Nucleotide | IMX129840-1 oligonucleotide probe |
| SEQ ID NO: 16 | Nucleotide | IMX129840-2 oligonucleotide primer |
| SEQ ID NO: 17 | Nucleotide | IMX129840-2 oligonucleotide primer |
| SEQ ID NO: 18 | Nucleotide | IMX129840-2 oligonucleotide probe |

What is claimed is:

1. An isolated cytokine polypeptide comprising an amino acid sequence selected from the group consisting of:
(a) the amino acid sequence of SEQ ID NO:2;
(b) the amino acid sequence of SEQ ID NO:4 or of SEQ ID NO:6;
(c) a fragment of an amino acid sequence of any of (b), said fragment comprising an amino acid sequence that begins between amino acid A through B and ends between amino acid Y through Z, wherein sets of values for A, B, Y, and Z are selected from the group consisting of: A =51, B=54, Y=66, and Z=68 of SEQ ID NO:4 or of SEQ ID NO:6; A=96, B=98, Y=109, and Z=121 of SEQ ID NO:4 or of SEQ ID NO:6; A=120, B=125, Y=140, and Z=144 of SEQ ID NO:4 or of SEQ ID NO:6; and A=152, B=155, Y=166, and Z=168 of SEQ ID NO:4 or of SEQ ID NO:6; wherein the fragment has IMX129840 cytokine polypeptide activity;
(d) a fragment of an amino acid sequence of any of (b)-(c) comprising at least 20 contiguous amino acids and having IMX129840 cytokine polypeptide activity;
(e) a fragment of an amino acid sequence of any of (b)-(c) comprising at least 30 contiguous amino acids and having IMX129840 cytokine polypeptide activity;
(f) a fragment of an amino acid sequence of any of (b)-(c) comprising Helix A and/or Helix D amino acid sequences and having IMX129840 cytokine polypeptide activity; and
(g) an amino acid sequence comprising at least 30 amino acids and sharing amino acid identity with the amino acid sequences of any of (b)-(f), wherein the percent amino acid identity is selected from the group consisting of: at least 97.5%, at least 99%, and at least 99.5%; and wherein a polypeptide consisting of said amino acid sequence has IMX129840 cytokine polypeptide activity.

2. The polypeptide of claim 1 comprising the amino acid sequence of SEQ ID NO:4.

3. The polypeptide of claim 1 comprising the amino acid sequence of SEQ ID NO:6.

4. An isolated nucleic acid encoding a polypeptide of claim 1.

5. The nucleic acid of claim 4 comprising a nucleotide sequence selected from the group consisting of:
(a) nucleotides 58 through 657 SEQ ID NO:1;
(b) nucleotides 141 through 740 of SEQ ID NO:3;
(c) nucleotides 141 through 740 of SEQ ID NO:5; and
(d) variants of (a)-(c).

6. An isolated genomic nucleic acid corresponding to the nucleic acid of any of claim 4.

7. An isolated nucleic acid encoding a polypeptide having IMX129840 cytokine polypeptide activity and comprising a nucleotide sequence that shares nucleotide sequence identity with the nucleotide sequences of the nucleic acid of claim 4, wherein the percent nucleotide sequence identity is selected from the group consisting of: at least 95%, at least 97.5%, at least 99%, and at least 99.5%.

8. An expression vector comprising at least one nucleic acid according to claim 4.

9. A recombinant host cell comprising at least one nucleic acid according to claim 4.

10. The recombinant host cell of claim 9, wherein the nucleic acid is integrated into the host cell genome.

11. A process for producing a polypeptide encoded by the nucleic acid of claim 4, comprising culturing a recombinant host cell under conditions promoting expression of said polypeptide, wherein the recombinant host cell comprises at least one nucleic acid of claim 4.

12. The polypeptide produced by the process of claim 11.

13. An isolated antibody that binds to the polypeptide of any of claim 12.

14. An isolated antibody wherein the antibody inhibits the activity of the polypeptide of claim 12.

15. A method for identifying compounds that alter IMX129840 cytokine polypeptide activity comprising
(a) mixing a test compound with the polypeptide of claim 12; and
(b) determining whether the test compound alters the IMX129840 cytokine polypeptide activity of said polypeptide.

16. A method for treating psoriasis comprising providing an antagonist of the polypeptide of claim 12.

17. A method for increasing epithelial barrier function in intestinal epithelia comprising providing an antagonist of the polypeptide of any of claim 12.

18. A method for treating an inflammatory condition of the intestine comprising providing an antagonist of the polypeptide of any of claim 12.

19. A method for increasing epithelial barrier function in lung epithelia comprising providing a polypeptide of claim 12.

20. A method for treating an inflammatory respiratory condition comprising providing a polypeptide of claim 12.
